(19) United States
(12) Patent Application Publication
(10) Pub. No.: US 2026/0306454 A1
(43) Pub. Date: Oct. 8, 2026

(54) N-N-DIMETHYLTRYPTAMINE (DMT) AND DMT ANALOG ORAL TRANSMUCOSAL FILM COMPOSITIONS, METHODS OF MAKING, AND METHODS OF USE THEREOF

(71) Applicant: Atai Therapeutics, Inc., New York, NY (US)

(72) Inventors: Vaibhav SAXENA, Overland Park, KS (US); Nadine PAIEMENT, Saint-Laurent (CA); Rubo ZHENG, Saint-Laurent (CA)

(21) Appl. No.: 19/637,329

(22) Filed: Apr. 2, 2026

Related U.S. Application Data
(60) Provisional application No. 63/782,175, filed on Apr. 2, 2025.

(51) Int. Cl. A61K 31/4045; A61K 9/00; A61K 47/02; A61K 47/18; A61K 47/22; A61K 47/24; A61K 47/32; A61K 47/38

(52) U.S. Cl. CPC A61K 31/4045; A61K 9/006; A61K 47/02; A61K 47/18; A61K 47/22; A61K 47/24; A61K 47/32; A61K 47/38

(57) ABSTRACT

The disclosure provides transmucosal film for delivering N,N-dimethyltryptamine (DMT) comprising an active layer comprising DMT or a pharmaceutically acceptable salt or prodrug thereof in a polymeric carrier matrix and a backing layer.

### CROSS-REFERENCE TO RELATED APPLICATIONS

This application claims the benefit of and priority to U.S. Provisional Application No. 63/782,175, filed Apr. 2, 2025, the contents of which are hereby incorporated by reference in their entirety for all purposes

### BACKGROUND OF THE INVENTION

Lysergic acid diethylamide (“LSD”), psilocybin and DMT are serotonergic agents often referred to as “classical hallucinogens” or “psychedelics,” and have the ability to induce qualitatively altered states of consciousness, such as euphoria, trance, transcendence of time and space, spiritual experiences, or dissolution of self-boundaries, while other effects such as sedation, narcosis, or excessive stimulation are only minimal. Chemically, serotonergic psychedelics are either phenylalkylamines or indoleamines, with the indoleamine class being divided into two subsets, ergolines and tryptamines.

Naturally occurring psychedelics, such as the DMT, which is contained in the South American shrub Psychotria viridis, psilocybin, which is contained in over 200 mushroom species, or mescaline, which is contained in the Peyote cactus of the American Southwest and Northern Mexico, have been used for centuries by indigenous cultures in ritualistic or sociocultural contexts, and in the context of religious sacraments. While an unspecific “healing” potential had been ascribed to the use of naturally occurring psychedelics in those settings, more scientific investigations into their potential therapeutic application for defined diseases had not been pursued until after the discovery of the synthetic ergoline lysergic acid diethylamide (“LSD”) in 1943.

With emerging knowledge about the serotonin system and its role in brain function, researchers began to specify the molecular activity of psychedelic drugs. However, how that activity translated into the observed therapeutic effects in mental disorders was less clear. Two main concepts were proposed: The first concept was coined “psycholytic therapy” and it emphasized the ability of psychedelics given at low doses to facilitate the loosening of psychological defensive mechanisms, which in combination with psychotherapy allows a deep introspective insight and the revival of traumata and their subsequent catharsis. The basic mechanism considered in the psycholytic approach was therefore the activation and deepening of the concomitant psychotherapeutic process, and it required multiple drug and therapy sessions. The second concept was coined as “psychedelic therapy” and it emphasized the ability of psychedelics given at relatively high single doses to induce so called “peak psychedelic experiences.” Peak experiences are predominantly characterized by the loss of judgment to time and space and the dissolution of ego boundaries, which often culminates in the experience of a blissful state and feelings of being a whole and harmonious existence in the cosmic unity. The basic mechanism considered in the psychedelic approach was therefore to produce a unique, overwhelming experience with an intuitive perception of psychological integration and harmony and subsequent self-improvements and enhanced joy in living and a sense of inner peace.

Although scientific research around the use of psychedelics for the treatment of mental disorders blossomed in the 1960s, there was also a rapidly growing recreational use of these substances, and soon psychedelics were depicted in the media as highly dangerous drugs of abuse. A perceived danger to the social order led to the passage of the United States Controlled Substances Act of 1970, under which LSD and other psychedelics were placed into the most restrictive category Schedule 1, which contains drugs deemed to have no medical use and a high potential for abuse. Very little progress was made regarding possible therapeutic uses of psychedelic drugs for the next 30 years.

Recently, interest in the field of psychedelic therapy has resurged, and classical psychedelics have shown preclinical and clinical promise in treating psychiatric disorders (Carhart-Harris and Goodwin, The Therapeutic Potential of Psychedelic Drugs: Past, Present and Future, Neuropsychopharmacology; 42, 2105-2113 (2017)). In particular, psilocybin has demonstrated significant improvement in a range of depression and anxiety rating scales in randomized double-blind studies (Griffiths et al., Psilocybin produces substantial and sustained decreases in depression and anxiety in patients with life-threatening cancer: a randomized double-blind trial, Journal of Psychopharmacology 30 (12), 1181-1197 (2016)).

DMT is also understood to hold therapeutic value as a psychedelic, with efficacy trials ongoing to assess the effect of DMT or DMT fumarate administered intravenously to patients with major depressive disorder (“MDD”). However, although the intrinsic properties of DMT make it an attractive possible medication, especially for neurological diseases and conditions, current therapeutic compositions and modes of administration complicate treatment and may not provide optimal therapeutic results. For example, when smoked or delivered intravenously, DMT has a very fast onset of action and a short duration of effect, which presents a challenge to determine a suitable administration regimen with appropriate dosage and frequency of administration of DMT to provide effective therapy for neurological diseases and conditions. This is especially true for neurological diseases and conditions which would benefit from the presence of therapeutic blood levels of DMT over a more extended period of time following administration than can be achieved with a single dose of DMT via injection or inhalation.

Therefore, a significant need exists for readily administrable medications of DMT to treat neurological diseases and conditions. Such medications, which maximize efficacy while enabling drug side effects to be effectively controlled, are of particular interest, especially if administrable via a convenient route, including self-administration.

### BRIEF DESCRIPTION OF THE DRAWINGS

FIG. 1 depicts a powder x-ray diffractogram of a 5 mg DMT oral transmucosal film.

FIG. 2 depicts a powder x-ray diffractogram of a 15 mg DMT oral transmucosal film.

FIG. 3 depicts results of an ex vivo permeation study conducted using pig mucosa for a 5 mg DMT oral transmucosal film.

FIG. 4 depicts results of an ex vivo permeation study conducted using pig mucosa for a 15 mg DMT oral transmucosal film.

### SUMMARY

In aspects, the present disclosure provides transmucosal compositions comprising:
(a) an active layer comprising N,N-dimethyltryptamine (DMT) or a pharmaceutically acceptable salt or prodrug thereof in a polymeric carrier matrix, and
(b) a backing layer,
wherein the active layer comprises about 0.5 wt % to about 20 wt % of DMT or a pharmaceutically acceptable salt or prodrug thereof, and
wherein the active layer comprises a plasticizer.

In aspects, the present disclosure provides methods of treating treatment resistant depression in a patient in need thereof, the method comprising administering the transmucosal compositions described herein. In embodiments, the administration provides a DMT Tₘₐₓ of about 0.3 h to about 2 h and a DMT Cₘₐₓ of about 0.1 ng/mL to about 10 ng/ml following administration. In embodiments, the administration provides a DMT Tₘₐₓ of about 0.3 h to about 2 h and a DMT AUCₗₐₛₜ of about 0.1 ng·h/mL to about 10 ng·h/mL following administration.

### DETAILED DESCRIPTION

The present disclosure provides transmucosal compositions comprising an active layer comprising DMT or a pharmaceutically acceptable salt or prodrug thereof in a polymeric carrier matrix and a backing layer to enhance transmucosal delivery of the DMT. In embodiments, the active layer of the present disclosure stabilizes DMT in amorphous form at low loading (e.g., less than 20 wt %, including about 0.5 wt % to about 20 wt %) and provides DMT release in a suitable dosage form, for example, a transmucosal dosage form. In embodiments, the backing layer of the present disclosure is layered on an active layer to provide a multilayer (e.g., bilayer) film in order to reduce or prevent erosion from the back side of the film when applied to the mucosal surface. In embodiments, more than one active layer may be layered on another (e.g., to provide multiple DMT-containing layers, such as a bilayer of active layer) optionally in combination with a backing layer (e.g., to provide a composition having multiple DMT-containing active layers with a backing layer).

### Definitions

For convenience, certain terms employed in the specification, examples and claims are collected here. Unless defined otherwise, all technical and scientific terms used in this disclosure have the same meanings as commonly understood by one of ordinary skill in the art to which this disclosure belongs.

The term “about” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example, in a list of numerical values such as “about 49, about 50, about 55, . . . ”, “about 50” means a range extending to less than half the interval(s) between the preceding and subsequent values, e.g., more than 49.5 to less than 50.5.

The term “patient” refers to a human.

The terms “treating,” “treat” and “treatment” as used herein with regard to a patient, refers to improving at least one symptom of the patient's disorder. Treating can be improving, or at least partially ameliorating a disorder or an associated symptom of a disorder.

The term “pharmaceutically acceptable” as used herein, refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “salts” as used herein embraces pharmaceutically acceptable salts commonly used to form alkali metal salts of free acids and to form addition salts of free bases. Salts include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Base addition salts include but are not limited to, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris-(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e. g., lysine and arginine dicyclohexylamine and the like. Examples of metal salts include lithium, sodium, potassium, magnesium, calcium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like. Examples of organic bases include lysine, arginine, guanidine, diethanolamine, choline and the like. Those skilled in the art will further recognize that acid addition salts may be prepared by reaction of the compounds with the appropriate inorganic or organic acid via any of a number of known methods.

The term “effective amount” or “therapeutically effective amount” as used interchangeably in this disclosure and refer to an amount of a compound, or a salt thereof, (or pharmaceutical composition containing the compound or salt) that, when administered to a patient, is capable of performing the intended result.

The term “carrier” or “vehicle” as used interchangeably herein encompasses carriers, excipients, adjuvants, and diluents or a combination of any of the foregoing, meaning a material, composition or vehicle, such as a liquid or solid filler, diluent, excipient, solvent or encapsulating material involved in carrying or transporting a pharmaceutical agent from one organ, or portion of the body, to another organ or portion of the body. In addition to the adjuvants, excipients and diluents known to one skilled in the art, the carrier includes nanoparticles of organic and inorganic nature.

### Compositions

In aspects, the present disclosure provides transmucosal compositions for delivering N,N-dimethyltryptamine (DMT) comprising (a) an active layer comprising DMT or a pharmaceutically acceptable salt or prodrug thereof in a polymeric carrier matrix, and (b) a backing layer, wherein the active layer comprises about 0.5 wt % to about 20 wt % of DMT or a pharmaceutically acceptable salt or prodrug thereof, and wherein the active layer comprises a plasticizer

### Active Layer

In embodiments, the transmucosal composition of the present disclosure comprises an active layer comprising about 0.5 wt % to about 20 wt % of N,N-dimethyltryptamine (DMT) or a pharmaceutically acceptable salt or prodrug thereof in a polymeric carrier matrix and about 70 wt % to about 90 wt % of the polymeric carrier matrix. In embodiments, the DMT in the composition is in an amorphous form as characterized by a powder x-ray diffractogram free of any discernable peaks that are attributable to crystalline DMT. In embodiments, the DMT in the composition is amorphous as characterized by a differential scanning calorimetry (DSC) thermogram lacking a sharp melting endotherm of crystalline DMT and/or lacking an indication of phase change. In embodiments, the indication of phase change is glass transition temperature. DMT and DMT analog compositions, methods of making and methods of use thereof are described in PCT Pub. No. WO2024/119075 and US Pat. App. Pub. No. 2023/0136824 A1, the contents of which are hereby incorporated by reference in their entireties for all purposes.

In embodiments, the active layer comprises about 0.5 wt % to about 30 wt %, including about 0.5 wt % to about 5 wt %, about 0.5 wt % to about 10 wt %, about 0.5 wt % to about 15 wt %, about 0.5 wt % to about 20 wt %, about 0.5 wt % to about 25 wt %, about 0.5 wt % to about 30 wt %, about 5 wt % to about 10 wt %, about 10 wt % to about 20 wt %, or about 20 wt % to about 30 wt % (including all values and ranges therebetween) of DMT or a pharmaceutically acceptable salt or prodrug thereof.

In embodiments, the active layer comprises DMT or a pharmaceutically acceptable salt or prodrug thereof of about 0.5 wt % to 20 wt % or about 1 wt % to 20 wt %, including about 0.5 wt %, about 1 wt %, about 2 wt %, about 3 wt %, about 4 wt %, about 5 wt %, about 6 wt %, about 7 wt %, about 8 wt %, about 9 wt %, about 10 wt %, about 11 wt %, about 12 wt %, about 13 wt %, about 14 wt %, about 15 wt %, about 16 wt %, about 17 wt %, about 18 wt %, about 19 wt %, or about 20 wt %, including all values and ranges therebetween. In embodiments, the active layer comprises about 1 wt %, about 2 wt %, about 3 wt %, about 5 wt %, about 7 wt %, about 9 wt %, about 12 wt %, about 15 wt %, or about 18 wt % of the DMT or a pharmaceutically acceptable salt or prodrug thereof.

In embodiments, the active layer of the present disclosure comprises about 1 mg to about 50 mg of DMT or a pharmaceutically acceptable salt thereof, for example, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, or about 50 mg, of DMT or a pharmaceutically acceptable salt or prodrug thereof, including all values and ranges therebetween. In embodiments, the active layer comprises about 1 mg to about 5 mg, about 5 mg to about 15 mg, about 15 mg to about 20 mg, or about 20 mg to about 30 mg DMT or a pharmaceutically acceptable salt or prodrug thereof. In embodiments, the active layer comprises about 5 mg, about 15 mg, about 20 mg, or about 30 mg DMT or a pharmaceutically acceptable salt or prodrug thereof.

In embodiments, more than one active layers may be layered on another (e.g., to provide multiple DMT-containing layers, such as a bilayer of active layer), optionally in combination with a backing layer (e.g., to provide a composition having multiple DMT-containing active layers with a backing layer). In embodiments, the composition comprises 1, 2, 3, or more active layers. In embodiments, each active layer of the multiple DMT-containing active layers comprises about 1 mg to about 20 mg of DMT or a pharmaceutically acceptable salt or prodrug thereof. In embodiments, each active layer of the multiple DMT-containing active layers comprises the same amount of DMT or a pharmaceutically acceptable salt or prodrug thereof or different amount of DMT or a pharmaceutically acceptable salt or prodrug thereof. In embodiments, the composition of the present disclosure comprises a first active layer and a second active layer, wherein the first active layer comprises about 1 mg to about 20 mg of DMT or a pharmaceutically acceptable salt or prodrug thereof and the second active layer comprises about 1 mg to about 20 mg of DMT or a pharmaceutically acceptable salt or prodrug thereof, including all values and ranges therebetween.

In embodiments, the active layer of the present disclosure is a mucoadhesive film. In embodiments, the active layer has a thickness of about 0.01 mm to about 1.5 mm. In embodiments, the active layer has a thickness of about 0.5 mm to about 1.5 mm. In embodiments, the active layer has a surface of about 2 cm² to about 10 cm², for example, about 2 cm², about 4 cm², about 6 cm², about 8 cm², or about 10 cm². In embodiments, the active layer comprises about 0.1 mg/cm² to about 10 mg/cm² amorphous DMT, for example, about 0.1 mg/cm², about 0.5 mg/cm², about 1 mg/cm², about 5 mg/cm², or about 10 mg/cm², including all values and ranges therebetween. In embodiments, the active layer comprises about 0.5 mg/cm² to about 5 mg/cm² the DMT or the pharmaceutically acceptable salt or prodrug thereof.

In aspects, the active layer of the present disclosure comprises a polymeric carrier matrix that is suitable for transmucosal delivery. In embodiments, the polymeric carrier matrix comprises a mucoadhesive polymer.

In embodiments, the polymeric carrier matrix comprises a cellulose derivative, a polyacrylic acid, a polyacrylate, a polyethylene oxide, polyvinyl pyrrolidone, poly(vinyl pyrrolidone-co-vinyl acetate), hydroxyethyl cellulose, hydroxypropyl cellulose (HPC), carboxymethyl cellulose (CMC), copovidone, hydroxypropylmethyl cellulose, polyvinyl alcohol, propylene glycol alginate ester, tragacanth, alginate, a gum, a soluble starch, gelatin, lectin, pectin, chitosan, or any combination thereof. Exemplary CMC includes, but is not limited to, Aqualon™ 7LF sodium CMC of 90.5 KDa having a solution viscosity (mPa·s) of 25-50 cps in a 2 wt % aqueous solution (Ashland Global Holdings Inc.). Exemplary copovidone includes, but is not limited to, Plasdone™ S-630 copovidone of 43 KDa having a solution viscosity (mPa s) of 25-31 cps in a 2 wt % aqueous solution (Ashland Global Holdings Inc.). In embodiments, the polymeric carrier matrix comprises HPC. In embodiments, the polymeric carrier matrix comprises HPC-L and HPC-GXF. Exemplary HPC-L and HPC-GXF are shown in Table 1.

TABLE 1
Exemplary HPC-L, HPC-GXF, and HPC-SSL

| | | Average molecular | Viscosity (2% Aq. |
|---|---|---|---|
| Vendor | Type | weight (Da) | Solution) (mPa · s) |
| Nisso | HPC-L | 140,000 | 6.0-10.0 |
| Klucel | HPC-GXF | 370,000 | 150.0-400.0 |

In embodiments, the polymeric carrier matrix stabilizes the DMT in an amorphous form within the mucoadhesive therapeutic composition.

In embodiments, the active layer comprises about 50 wt % to about 90 wt %, including about 50 wt %, about 55 wt %, about 60 wt %, about 65 wt %, about 70 wt %, about 75 wt %, about 80 wt %, or about 90 wt % of the polymeric carrier matrix. In embodiments, the active layer comprises about 70 wt % to about 90 wt % of the polymeric carrier matrix. In embodiments, the active layer comprises about 70 wt % to about 90 wt % of the polymeric carrier matrix comprising Plasdone™ S-630 copovidone having an average formula weight of about 43 KDa, wherein a 2 wt % aqueous solution of the copovidone provides a solution viscosity of about 25 to about 31 mPa·S, HPC having an average formula weight of about 140 KDa, wherein a 2 wt % aqueous solution of the HPC provides a solution viscosity of about 6 to about 10 mPa·S, and HPC having an average formula weight of about 370 KDa, wherein a 2 wt % aqueous solution of the HPC provides a solution viscosity of about 150 to about 400 mPa·S.

In embodiments, the active layer comprises a plasticizer. In embodiments, the active layer comprises about 1 wt % to about 50 wt %, for example, about 1 wt % to about 10 wt %, about 10 wt % to about 20 wt %, about 20 wt % to about 30 wt %, about 30 wt % to about 40 wt %, about 40 wt % to about 50 wt % of the plasticizer. In embodiments, the active layer comprises about 10 wt % to 20 wt % of the plasticizer. In embodiments, the plasticizer is a polyethylene glycol (PEG), propylene glycol, glycerol, triacetin, castor oil, or any combination thereof.

In embodiments, the active layer comprises an anti-foaming agent. In embodiments, the active layer comprises about 0.01 wt % to about 10 wt %, for example, about 0.01 wt % to about 0.1 wt %, about 0.05 wt % to about 1 wt %, about 0.1 wt % to about 1 wt %, or about 1 wt % to about 10 wt % of the anti-foaming agent. In embodiments, the active layer comprises about 0.05 wt % to about 1 wt % of the anti-foaming agent. In embodiments, the anti-foaming agent is silicone, polyglycol, or any combination thereof. In embodiments, the anti-foaming agent is simethicone.

In embodiments, the active layer comprises a buffering agent. In embodiments, the active layer comprises about 0.01 wt % to about 10 wt %, for example, about 0.01 wt % to about 0.1 wt %, about 0.1 wt % to about 1 wt %, or about 1 wt % to about 10 wt % of the buffering agent. In embodiments, the active layer comprises about 0.1 wt % to about 1 wt % of the buffering agent. In embodiments, the buffering agent comprises one or more of citric acid, tartaric acid, fumaric acid, succinic acid, malic acid, sodium citrate, sodium tartrate, sodium succinate, sodium fumarate, sodium hydroxide, or any combination thereof. In embodiments, the active layer comprises a buffering agent, and/or a saliva stimulating agent. In embodiments, the buffering agent is used with a pH adjusting agent, such as sodium hydroxide. In embodiments, the saliva stimulating agent comprises one or more of citric acid, malic acid, lactic acid, ascorbic acid, tartaric acid, or any combination thereof.

In embodiments, the active layer comprises a permeation enhancer. In embodiments, the active layer comprises about 0.1 wt % to about 10 wt %, about 0.1 wt % to about 1 wt %, about 1 wt % to about 5 wt %, or about 5 wt % to about 10 wt %, of the permeation enhancer. In embodiments, the active layer comprises about 0.1 wt % to 1 wt % of the permeation enhancer. In embodiments, the permeation enhancer comprises d-α-tocopheryl polyethylene glycol succinate (“Vitamin E TPGS”), a bile salt, cetylpyridinium chloride (CPC), sodium lauryl sulfate (SLS), Tween 80, L-menthol, dimethyl sulfoxide (DMSO), oleic alcohol, oleic acid, oleyl oleate, levulinic acid, propylene glycol, dipropylene glycol, ethanol, or any combination thereof. In embodiments, the permeation enhancer comprise Vitamin E TPGS. In embodiments, the composition comprises other stability enhancers including antioxidants or chelators. In embodiments, the stability enhancer comprises one or more of α-tocopherol, tocopherol acetate, L-Glutathione, L-cysteine, ascorbic acid, ascorbyl palmitate, propyl gallate, butylated hydroxytoluene (BHT), butylated hydroxyanisole (BHA), Tocobiol, Ethylenediaminetetraacetic acid (EDTA), or any combination thereof.

In embodiments, the active layer comprises an antioxidant. In embodiments, the active layer comprises at least about 2 wt % of the antioxidant. In embodiments, the active layer comprises about 0.1 wt % to about 10 wt %, for example, for example, about 0.1 wt % to about 1 wt %, about 2 wt % to about 6 wt %, or about 1 wt % to about 10 wt % of the antioxidant. In embodiments, the permeation enhancer comprises both permeation enhancing and antioxidant properties. For example, vitamin E may include both permeation enhancing and antioxidant characteristics. In embodiments, the active layer comprises about 2 wt % to about 10 wt %, about 4 wt % to about 8 wt %, or about 5 wt % to about 6 wt % of the permeation enhancer and the antioxidant.

In embodiments, the active layer comprises a sweetening agent and/or a flavoring agent. In embodiments, the active layer comprises about 0.01 wt % to about 5 wt %, about 0.01 wt % to about 0.1 wt %, about 0.01 wt % to about 1 wt %, about 0.1 wt % to about 1 wt %, or about 1 wt % to about 5 wt % of the sweetening agent and/or a flavoring agent. In embodiments, the sweetening agent comprises one or more of advantame, sucrose, dextrose, fructose, glucose, maltose, maltitol, saccharin, sucralose, neotame, cyclamate, aspartame, acesulfame-K, or any combination thereof. In embodiments, the flavoring agent comprises one or more of lime flavor, lemon flavor, peppermint oil, cinnamon oil, vanilla extract, menthol, L-menthol, or any combination thereof. In embodiments, the active layer comprises advantame.

In embodiments, the active layer comprises a coloring agent. In embodiments, the active layer comprises about 0.1 wt % to about 10 wt %, about 0.1 wt % to about 1 wt %, about 0.1 wt % to about 1.5 wt %, about 1 wt % to about 5 wt %, or about 5 wt % to about 10 wt % of the coloring agent. In embodiments, the coloring agent comprises one or more of D&C or FD&C red, yellow, green, blue, iron oxide red, iron oxide yellow, titanium dioxide, or any combination thereof.

In embodiments, the active layer composition is as shown in Table 2.

TABLE 2
Active layer composition

| wt % | Components |
|---|---|
| about 1 to about 20 | DMT (N,N-dimethyltryptamine) |
| about 10 to about 20 | plasticizer |
| about 70 to about 90 | polymeric carrier matrix |
| about 0.05 to about 1 | anti-foaming agent |
| about 0.01 to about 1 | sweetening agent |
| about 0.1 to about 1.5 | coloring agent |
| about 0.1 to about 1 | buffering agent |
| about 0.1 to about 1 | permeation enhancer |
| about 2 to about 6 | antioxidant |

*the percentage is relative to the weight of the active layer.

In embodiments, the active layer composition is as shown in Table 3.

TABLE 3
Active layer composition

| wt % | Components |
|---|---|
| about 2 to about 3 | DMT (N,N-dimethyltryptamine) |
| about 10 to about 20 | PEG300 and triacetin |
| at least about 70 | polymeric carrier matrix: |
| | (i) Copovidone, |
| | (ii) HPC having an average formula weight of about 140 |
| | KDa, wherein a 2 wt % aqueous solution of the HPC |
| | provides a solution viscosity of about 6 to about 10 |
| | mPa · S, |
| | (iii) HPC having an average formula weight of about 370 |
| | KDa, wherein a 2 wt % aqueous solution of the HPC |
| | provides a solution viscosity of about 150 to about 400 |
| | mPa · S; and |
| | (iv) Sodium carboxymethylcellulose. |
| about 0.05 to about 1 | Simethicone |
| about 0.1-about 1 | TPGS (vitamin E) |
| about 1-about 5 | L-Glutathione |

*the percentage is relative to the weight of the active layer.

In embodiments, the active layer composition is as shown in Table 4.

TABLE 4
Active layer film composition

| wt % | Components |
|---|---|
| about 6 to about 9 | DMT (N,N-dimethyltryptamine) |
| about 10 to about 20 | PEG300 and triacetin |
| at least about 70 | polymeric carrier matrix: |
| | (i) Copovidone, |
| | (ii) HPC having an average formula weight of about 140 |
| | KDa, wherein a 2 wt % aqueous solution of the HPC |
| | provides a solution viscosity of about 6 to about 10 |
| | mPa · S, |
| | (iii) HPC having an average formula weight of about 370 |
| | KDa, wherein a 2 wt % aqueous solution of the HPC |
| | provides a solution viscosity of about 150 to about 400 |
| | mPa · S; and |
| | (iv) Sodium carboxymethylcellulose. |
| about 0.05 to about 1 | Simethicone |
| about 0.1-about 1 | TPGS (vitamin E) |
| about 1-about 5 | L-Glutathione |

*the percentage is relative to the weight of the active layer.

In embodiments, the active layer composition is as shown in Table 5.

TABLE 5
Active layer film composition

| wt % | Components |
|---|---|
| about 1 to about 20 | DMT (N,N-dimethyltryptamine) |
| about 1 to about 10 | Hydroxypropyl cellulose (Klucel GXF) |
| about 20 to about 40 | Hydroxypropyl cellulose (Nisso HPC-L) |
| about 20 to about 40 | Copovidone (Plasdone S-630) |
| about 1 to about 5 | Sodium carboxymethylcellulose |
| about 0.1 to about 1 | Sodium hydroxide |
| about 0.05 to about 1 | Simethicone |
| about 1 to about 10 | L-Glutathione |
| about 0.1 to about 1 | TPGS (vitamin E) |
| about 5 to about 15 | Polyethylene Glycol 300 (PEG-300) |
| about 1 to about 10 | Triacetin |
| about 0.01 to about 0.1 | Advantame |
| about 0.1 to about 1 | Sucralose |
| about 0.1 to about 1 | Arome Type Suppresseur amertume |
| about 0.1 to about 1 | Evospray natural lime flavor |
| about 0.01 to about 0.1 | FD&C Yellow# 6 powder |
| about 0.01 to about 0.1 | D&C Yellow# 10 Aluminum Lake |
| about 0.01 to about 0.1 | FD&C Blue# 1 Aluminum Lake |
| about 0.1 to about 1 | Titanium dioxide |

*the percentage is relative to the weight of the active layer.

In embodiments, the active layer of the present disclosure has a pH of about 7.5 to about 9, including about 8 to about 9, about 8.5 to about 9, or about 8.6. The pH can be measured by a method known to one skilled in the art, such as a standardized method according to United States Pharmacopeial Convention (USP) (e.g., a pH meter or pH electrode).

### Backing Layer

In embodiments, the transmucosal composition comprises a backing layer and an active layer (e.g., a DMT mucoadhesive film) as described herein. In embodiments, the backing layer forms the back side (i.e., the non-adhesive side) of the transmucosal composition of the present disclosure. In embodiments, the backing layer is adjacent to the DMT mucoadhesive film. In embodiments, the backing layer is adapted to reduce or prevent DMT from being transported across the backing layer while the mucoadhesive therapeutic product is being administered. In embodiments, the backing layer is substantially impermeable to diffusion of DMT to the back side of the transmucosal bilayer film. In embodiments, the backing layer serves as a protective barrier to prevent, substantially prevent or reduce the amount of saliva contacting the DMT active agent and/or erosion of the back side of transmucosal bilayer film.

In embodiments, the backing layer comprises a polymeric matrix and a coloring agent. In embodiments, the backing layer comprises about 60 wt % to about 90 wt %, including about 60 wt %, about 65 wt %, about 70 wt %, about 75 wt %, about 80 wt %, or about 90 wt % of the polymeric matrix. In embodiments, the backing layer comprises carboxymethyl cellulose (CMC), hydroxypropyl cellulose (HPC), sodium carboxymethylcellulose, polyethylene glycol (PEG), copovidone (a copolymer of N-vinyl-2-pyrrolidone and vinyl acetate), or any combination thereof. Exemplary CMC includes, but is not limited to, Aqualon™ 7LF PH sodium CMC of 90.5 KDa having a solution viscosity (mPa s) of 25-50 cps in a 2 wt % aqueous solution (Ashland Global Holdings Inc.). Exemplary copovidone includes, but is not limited to, Plasdone™ S-630 copovidone of 43 KDa having a solution viscosity (mPa s) of 25-31 cps in a 2 wt % aqueous solution (Ashland Global Holdings Inc.). Plasdone™ S-630 copovidone is a 60:40 linear random copolymer of N-vinyl-2-pyrrolidone and vinyl acetate. Exemplary PEG includes, but is not limited to, PEG300 of 300 Da having a solution viscosity (mPa s) of 5.4-6.4 cSt. Exemplary HPC is as shown in Table 1.

In embodiments, the backing layer comprises about 1 wt % to about 10 wt %, including about 1 wt %, about 3 wt %, about 5 wt %, about 7 wt %, about 9 wt %, or about 10 wt % of the coloring agent. In embodiments, the backing layer comprises about 1 wt % to about 3 wt % of the coloring agent. In embodiments, the backing layer comprises titanium dioxide. In embodiments, the backing layer comprises sodium hydroxide. In embodiments, the backing layer comprises about 0.1 wt % to about 5 wt %, including about 0.5 wt % to about 5 wt %, about 0.5 wt % to about 2 wt % of sodium hydroxide (e.g., sodium hydroxide NF solution 50% (w/w)).

In embodiments, the backing layer composition is as shown in Table 6.

TABLE 6
Backing layer film composition

| wt % | Components |
|---|---|
| about 60 to about 90 | Polymeric matrix |
| about 10 to about 20 | PEG |
| about 1 to about 3 | Titanium dioxide (TiO₂) |
| about 0 to about 7 | other |

*the percentage is relative to the weight of the backing layer.

In embodiments, the backing layer composition is as shown in Table 7.

TABLE 7
Backing layer film composition

| wt % | Components |
|---|---|
| about 60 to about 80 | Hydroxypropyl Cellulose (e.g., Nisso HPC-L) |
| about 3 to about 10 | Copovidone (e.g., Plasdone S-630) |
| about 5 to about 10 | Sodium Carboxymethylcellulose (e.g., Aqualon CMC 7LF PH) |
| about 10 to about 20 | Polyethylene Glycol (e.g., PEG 300) |
| about 1 to about 3 | Titanium dioxide (TiO₂) |
| about 0.5 to about 2 | Sodium Hydroxide NF solution 50% (w/w) |
| about 0 to about 7 | other |

*the percentage is relative to the weight of the backing layer.

In embodiments, the backing layer composition is as shown in Table 8.

TABLE 8
Backing layer film composition

| wt % | Components |
|---|---|
| about 68.4 | Nisso HPC-L |
| about 7.8 | CMC (ashland 7LF) |
| about 6.0 | Plasdone S-630 |
| about 14.3 | PEG300 |
| about 2.1 | Titanium dioxide (TiO₂) |
| about 1.4 | NaOH 50% |

*the percentage is relative to the weight of the backing layer.

### Transmucosal Composition

In embodiments, the transmucosal composition of the present disclosure comprises an active layer of about 70 wt % to about 90 wt %, including about 70 wt %, about 75 wt %, about 80 wt %, or about 85 wt %, to about 90 wt %, of the composition, including all values and ranges therebetween. In embodiments, the transmucosal bilayer film composition of the present disclosure comprises a backing layer of about 10 wt % to about 30 wt %, including about 10 wt %, about 15 wt %, about 20 wt %, or about 25 wt %, to about 30 wt % of the composition, including all values and ranges therebetween. In embodiments, the transmucosal composition of the present disclosure comprises an active layer comprising DMT or a pharmaceutically acceptable salt thereof and a backing layer at a ratio by weight of about 2:1 to about 5:1, for example, about 2:1, about 3:1, about 4:1, or about 5:1, including all values and ranges therebetween. In embodiments, the transmucosal composition is in the form of a transmucosal film.

In embodiments, the transmucosal film composition is as shown in the Table 9

TABLE 9
Transmucosal film composition

| | |
|---|---|
| wt % | Components |

an active layer comprising:

| | |
|---|---|
| about 1 to about 20 | N,N-Dimethyltryptamine (DMT) |
| about 10 to about 20 | PEG300 and triacetin |
| about 20 to about 40 | Copovidone |
| about 20 to about 40 | Hydroxypropyl cellulose (Nisso HPC-L) |
| about 1 to about 10 | Hydroxypropyl cellulose (Klucel GXF) |
| about 1 to about 5 | Sodium carboxymethylcellulose |
| about 0.05 to about 1 | simethicone |
| about 1 to about 10 | L-Glutathione |
| about 0.1 to about 1 | Sodium hydroxide |
| about 0.01 to about 0.1 | Advantame |
| about 0.1 to about 1 | Sucralose |
| about 0.1 to about 1.5 | FD&C Yellow 6 powder, D&C Yellow# 10 Aluminum Lake, FD&C |
| | Blue# 1 Aluminum Lake, and Titanium dioxide |
| about 0.1 to about 1 | Arome Type Suppresseur Amertume |
| about 0.1 to about 1 | Evospray Natural Lime Flavor |
| about 0.1 to about 1 | Vitamin E TPGS |

a backing layer comprising:

| | |
|---|---|
| about 3 to about 10 | Copovidone (Plasdone S-630) |
| about 60 to about 80 | Hydroxypropyl Cellulose (Nissa HPC-L) |
| about 5 to about 10 | Sodium Carboxymethylcellulose (Aqualon CMC 7LF PH) |
| about 10 to about 20 | Polyethylene Glycol (PEG 300) |
| about 1 to about 3 | Titanium Dioxide |
| about 0.5 to about 2 | Sodium Hydroxide NF solution 50% (W/W) |

*the percentage is relative to the weight of the active layer and backing layer respectively.

In embodiments, the transmucosal film of the present disclosure shows a folding endurance, e.g., at least about 5 folds, at least about 10 folds, at least about 20 folds, at least about 30 folds, at least about 40 folds, at least about 50 folds, at least about 60 folds, at least about 70 folds, at least about 80 folds, at least about 90 folds, at least about 100 folds, at least about 110 folds, at least about 120 folds, at least about 130 folds, at least about 140 folds, at least about 150 folds, at least about 160 folds, at least about 170 folds, at least about 180 folds, at least about 190 folds, at least about 200 folds, at least about 210 folds, at least about 220 folds, at least about 230 folds, at least about 240 folds, at least about 250 folds, at least about 260 folds, at least about 270 folds, at least about 280 folds, at least about 290 folds, at least about 300 folds, at least about 310 folds, at least about 320 folds, at least about 330 folds, at least about 340 folds, at least about 350 folds, at least about 360 folds, at least about 370 folds, at least about 380 folds, at least about 390 folds, at least about 400 folds at least about 410 folds, at least about 420 folds, at least about 430 folds, at least about 440 folds, at least about 450 folds, at least about 460 folds, at least about 470 folds, at least about 480 folds, at least about 490 folds, or at least about 500 folds, or more folds on a folding endurance test known to one skilled in the art (e.g., Mecmesin MultiTest-dV-Motorised Test).

In embodiments, the composition is suitable for transmucosal application. In embodiments, the composition is suitable for buccal, sublingual, gingival, on the tongue, nasal, vaginal, or a rectal application. In embodiments, the film is placed in the oral cavity with the backing layer facing away from the patient's skin (i.e., on the side of the film opposite that which is put into contact with the patient's skin).

In embodiments, the administration of the composition to the oral mucosa of a patient in need thereof provide residence time of DMT of about 5 min to about 20 min, for example, about 5 min, about 10 min, about 15 min, about 20 min, including any values or ranges therebetween following administration.

In embodiments, the administration of the composition to the oral mucosa of a patient in need thereof provide a Tₘₐₓ of DMT of about 0.1 h to about 4 h (such as about 0.3 h to about 2 h), for example, about 0.1 h, about 0.2 h, about 0.3 h, about 0.4 h, about 0.5 h, about 0.6 h, about 0.7 h, about 0.8 h, about 0.9 h, about 1.0 h, about 1.2 h, about 1.4 h, about 1.6 h, about 1.8 h, about 2.0 h, about 2.2 h, about 2.4 h, about 2.6 h, about 2.8 h, about 3.0 h, about 3.2 h, about 3.4 h, about 3.6 h, about 3.8 h, or about 4.0 h, including any values or ranges therebetween following administration.

In embodiments, the administration of the composition to the oral mucosa of a patient in need thereof provide a Tₘₐₓ of DMT of about 10 min to about 240 min, for example, about 10 min, about 15 min, about 20 min, about 25 min, about 30 min, about 35 min, about 40 min, about 45 min, about 50 min, about 55 min, about 60 min, about 65 min, about 70 min, about 75 min, about 80 min, about 85 min, about 90 min, about 95 min, about 100 min, about 105 min, about 110 min, about 115 min, about 120 min, about 125 min, about 130 min, about 135 min, about 140 min, about 145 min, about 150 min, about 155 min, about 160 min, about 165 min, about 170 min, about 175 min, about 180 min, about 185 min, about 190 min, about 195 min, about 200 min, about 205 min, about 210 min, about 215 min, about 220 min, about 225 min, about 230 min, about 235 min, or about 240 min, including any values or ranges therebetween following administration.

In embodiments, the administration of the composition to the oral mucosa of a patient in need thereof provides a Cₘₐₓ of DMT of about 0.1 ng/ml to about 10 ng/ml, for example, about 0.1 ng/mL, about 0.5 ng/ml, about 1 ng/ml, about 5 ng/ml, or about 10 ng/ml, including any values or ranges therebetween following administration.

In embodiments, the administration of the composition to the oral mucosa of a patient in need thereof provides a AUCₗₐₛₜ of DMT of about 0.1 ng·h/mL to about 10 ng·h/mL, for example, about 0.1 ng·h/mL, about 0.5 ng·h/mL, about 1 ng·h/mL, about 5 ng·h/mL, or about 10 ng·h/mL, including any values or ranges therebetween following administration.

### Dosing Regimens

In embodiments, about 1 mg to about 50 mg of DMT free base or an equivalent dose of the pharmaceutically acceptable salt thereof is administered to a patient in need thereof. In embodiments, the therapeutically effective amount comprises a dose ranging about 1 mg to about 30 mg DMT free base or an equivalent dose of the pharmaceutically acceptable salt thereof. In embodiments, the therapeutically effective amount comprises a dose ranging about 1 mg to about 50 mg, about 1 mg to about 40 mg, about 1 mg to about 30 mg, about 1 mg to about 20 mg, about 5 mg to about 20 mg, about 1 mg to about 5 mg, about 5 mg to about 15 mg, about 15 mg to about 20 mg, or about 20 mg to about 30 mg DMT free base or an equivalent dose of the pharmaceutically acceptable salt thereof. In embodiments, the therapeutically effective amount comprises a dose of about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, or about 50 mg, or more DMT free base or an equivalent dose of the pharmaceutically acceptable salt thereof. In embodiments, the therapeutically effective amount comprises a dose ranging about 1 mg to about 20 mg DMT free base or an equivalent dose of the pharmaceutically acceptable salt thereof. In embodiments, the therapeutically effective amount comprises a dose of about 1 mg to about 5 mg, about 5 mg to about 15 mg, about 15 mg to about 20 mg, or about 20 mg to about 30 mg of DMT free base or an equivalent dose of the pharmaceutically acceptable salt thereof. In embodiments, the therapeutically effective amount comprises a dose of about 5 mg, about 15 mg, about 20 mg, or about 30 mg DMT free base or an equivalent dose of the pharmaceutically acceptable salt thereof.

The effective amounts may be provided as a single dose or on regular schedule, i.e., on a daily, weekly, monthly, or yearly basis or an irregular schedule with varying administration days, weeks, months, etc. In embodiments, a patient is administered the composition of the present disclosure multiple times over a set a period of time. In embodiments, the patient is administered the composition once every time period of between about 2 weeks and about 12 months for a set treatment period. In embodiments, the patient is administered the composition once every time period of between about 2 weeks and about 6 months for a set treatment period. In embodiments, the patient is administered the composition once every about 2 weeks, about 3 weeks, about 4 weeks, about 2 months, about 3 months, about 4 months, about 5 months, about 6 months, about 7 months, about 8 months, about 9 months, about 10 months, about 11 months, or about 12 months for a set treatment period. In embodiments, the patient is administered the composition once every about 2 weeks for a set treatment period. In embodiments, the patient is administered the composition once every month for a set treatment period. In embodiments, the patient is administered the composition once every about 6 weeks for a set treatment period. In embodiments, the patient is administered the composition once every about 2 months for a set treatment period. In embodiments, the patient is administered the composition once every about 3 months for a set treatment period. In embodiments, the patient is administered the composition once every about 6 months for a set treatment period.

To reduce the occurrence of possible side effect associated with the dose, an effective amount may be provided as a split dose, where the single dose is split into two doses, that are administered apart, usually over several hours. For example, a single dose may be split into two doses, administered 1 hour apart, 2 hours apart, 3 hours apart, 4 hours apart, 5 hours apart, 6 hours apart, 7 hours apart, 8 hours apart, or more. Alternatively, the therapeutically effective amount to be administered may vary. In aspects, the therapeutically effective amount for the first dose is higher than the therapeutically effective amount for one or more of the subsequent doses. In aspects, the therapeutically effective amount for the first dose is lower than the therapeutically effective amount for one or more of the subsequent doses. Equivalent dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every 2 weeks, about every 3 weeks, about every month, about every 2 months, about every 3 months and about every 6 months. The number and frequency of dosages corresponding to a completed course of therapy will be determined according to the judgment of a health-care practitioner.

In embodiments, the administration of the composition of the present disclosure is buccal, sublingual, gingival, on the tongue, nasal, vaginal, or a rectal administration. In embodiments, the administration of the composition of the present disclosure is buccal or sublingual.

In embodiments, the oral administration of the compositions of the present disclosure provides a Tₘₐₓ of DMT of about 0.1 h to about 4 h (such as about 0.3 h to about 2 h), for example, about 0.1 h, about 0.2 h, about 0.3 h, about 0.4 h, about 0.5 h, about 0.6 h, about 0.7 h, about 0.8 h, about 0.9 h, about 1.0 h, about 1.2 h, about 1.4 h, about 1.6 h, about 1.8 h, about 2.0 h, about 2.2 h, about 2.4 h, about 2.6 h, about 2.8 h, about 3.0 h, about 3.2 h, about 3.4 h, about 3.6 h, about 3.8 h, or about 4.0 h, including any values or ranges therebetween following administration of the composition.

In embodiments, the oral administration of the compositions of the present disclosure provides a Tₘₐₓ of DMT of about 10 min to about 240 min, for example, about 10 min, about 15 min, about 20 min, about 25 min, about 30 min, about 35 min, about 40 min, about 45 min, about 50 min, about 55 min, about 60 min, about 65 min, about 70 min, about 75 min, about 80 min, about 85 min, about 90 min, about 95 min, about 100 min, about 105 min, about 110 min, about 115 min, about 120 min, about 125 min, about 130 min, about 135 min, about 140 min, about 145 min, about 150 min, about 155 min, about 160 min, about 165 min, about 170 min, about 175 min, about 180 min, about 185 min, about 190 min, about 195 min, about 200 min, about 205 min, about 210 min, about 215 min, about 220 min, about 225 min, about 230 min, about 235 min, or about 240 min, including any values or ranges therebetween following administration of the composition.

In embodiments, the oral administration of the compositions of the present disclosure provides a T_{1/2} of DMT of about 0.1 h to about 2 h (such as about 0.3 h to about 1 h), for example, about 0.1 h, about 0.2 h, about 0.3 h, about 0.4 h, about 0.5 h, about 0.6 h, about 0.7 h, about 0.8 h, about 0.9 h, about 1.0 h, about 1.2 h, about 1.4 h, about 1.6 h, about 1.8 h, or about 2.0 h, including any values or ranges therebetween following administration of the composition.

In embodiments, the oral administration of the compositions of the present disclosure provides a Tₘₐₓ of DMT-NO of about 1 h to about 4 h, for example, about 1.0 h, about 1.2 h, about 1.4 h, about 1.6 h, about 1.8 h, about 2.0 h, about 2.2 h, about 2.4 h, about 2.6 h, about 2.8 h, about 3.0 h, about 3.2 h, about 3.4 h, about 3.6 h, about 3.8 h, or about 4.0 h, including any values or ranges therebetween following administration of the composition.

In embodiments, the oral administration of the compositions of the present disclosure provides a Tₘₐₓ of DMT-NO of about 60 min to about 240 min, for example, about 60 min, about 65 min, about 70 min, about 75 min, about 80 min, about 85 min, about 90 min, about 95 min, about 100 min, about 105 min, about 110 min, about 115 min, about 120 min, about 125 min, about 130 min, about 135 min, about 140 min, about 145 min, about 150 min, about 155 min, about 160 min, about 165 min, about 170 min, about 175 min, about 180 min, about 185 min, about 190 min, about 195 min, about 200 min, about 205 min, about 210 min, about 215 min, about 220 min, about 225 min, about 230 min, about 235 min, or about 240 min, including any values or ranges therebetween following administration of the composition.

In embodiments, the oral administration of the compositions of the present disclosure provides a T_{1/2} of DMT-NO of about 1 h to about 8 h, for example, about 1 h, about 2 h, about 3 h, about 4 h, about 5 h, about 6 h, about 7 h, or about 8 h, including any values or ranges therebetween following administration of the composition.

In embodiments, the oral administration of the compositions of the present disclosure provides a Tₘₐₓ of IAA of about 1 h to about 4 h, for example, about 1.0 h, about 1.2 h, about 1.4 h, about 1.6 h, about 1.8 h, about 2.0 h, about 2.2 h, about 2.4 h, about 2.6 h, about 2.8 h, about 3.0 h, about 3.2 h, about 3.4 h, about 3.6 h, about 3.8 h, or about 4.0 h, including any values or ranges therebetween following administration of the composition.

In embodiments, the oral administration of the compositions of the present disclosure provides a Tₘₐₓ of IAA of about 60 min to about 240 min, for example, about 60 min, about 65 min, about 70 min, about 75 min, about 80 min, about 85 min, about 90 min, about 95 min, about 100 min, about 105 min, about 110 min, about 115 min, about 120 min, about 125 min, about 130 min, about 135 min, about 140 min, about 145 min, about 150 min, about 155 min, about 160 min, about 165 min, about 170 min, about 175 min, about 180 min, about 185 min, about 190 min, about 195 min, about 200 min, about 205 min, about 210 min, about 215 min, about 220 min, about 225 min, about 230 min, about 235 min, or about 240 min, including any values or ranges therebetween following administration of the composition.

In embodiments, the oral administration of the compositions of the present disclosure provides a T_{1/2} of IAA of about 1 h to about 8 h, for example, about 1 h, about 2 h, about 3 h, about 4 h, about 5 h, about 6 h, about 7 h, or about 8 h, including any values or ranges therebetween following administration of the composition.

In embodiments, the oral administration of the composition provides a Cₘₐₓ of DMT of about 0.1 ng/mL to about 10 ng/ml, for example, about 0.1 ng/ml, about 0.5 ng/mL, about 1 ng/mL, about 5 ng/mL, or about 10 ng/mL, including any values or ranges therebetween following administration.

In embodiments, the oral administration of the composition provides a Cₘₐₓ of DMT-NO of about 0.1 ng/ml to about 20 ng/mL, for example, about 0.1 ng/mL, about 0.5 ng/ml, about 1 ng/mL, about 5 ng/mL, about 10 ng/ml, about 15 ng/ml, or about 20 ng/mL, including any values or ranges therebetween, following administration of the composition.

In embodiments, the oral administration of the composition provides a Cₘₐₓ of IAA of about 10 ng/ml to about 2,000 ng/ml, for example, about 10 ng/ml, about 50 ng/ml, about 100 ng/mL, about 500 ng/mL, about 1,000 ng/mL, about 1,500 ng/ml, or about 2,000 ng/ml, including any values or ranges therebetween, following administration of the composition.

In embodiments, the oral administration of the composition provides a AUCₗₐₛₜ of DMT of about 0.1 ng·h/mL to about 10 ng·h/mL, for example, about 0.1 ng·h/mL, about 0.5 ng·h/mL, about 1 ng·h/mL, about 5 ng·h/mL, or about 10 ng·h/mL, including any values or ranges therebetween, following administration of the composition.

In embodiments, the oral administration of the composition provides a AUCₗₐₛₜ of DMT-NO of about 0.1 ng h/mL to about 10 ng·h/mL, for example, about 0.1 ng·h/mL, about 0.5 ng·h/mL, about 1 ng·h/mL, about 5 ng·h/mL, or about 10 ng·h/mL, including any values or ranges therebetween, following administration of the composition.

In embodiments, the oral administration of the composition provides a AUCₗₐₛₜ of IAA of about 100 ng·h/mL to about 10,000 ng h/mL, for example, about 100 ng h/mL, about 500 ng·h/mL, about 1,000 ng·h/mL, about 5,000 ng·h/mL, or about 10,000 ng·h/mL, including any values or ranges therebetween, following administration of the composition.

In embodiments, the compositions of the present disclosure comprise about 1 mg to about 50 mg of DMT or a pharmaceutically acceptable salt thereof, wherein the administration provides a DMT Tₘₐₓ of about 0.3 h to about 2 h and a DMT Cₘₐₓ of about 0.1 ng/ml to about 10 ng/ml following administration. In embodiments, the DMT Tₘₐₓ is about 0.3 h, about 0.4 h, about 0.5 h, about 0.6 h, about 0.7 h, about 0.8 h, about 0.9 h, about 1.0 h, about 1.2 h, about 1.4 h, about 1.6 h, about 1.8 h, about 2.0 h, about 2.2 h, about 2.4 h, about 2.6 h, about 2.8 h, about 3.0 h, about 3.2 h, about 3.4 h, about 3.6 h, about 3.8 h, or about 4.0 h, including any values or ranges therebetween following administration of the composition. In embodiments, the DMT Cₘₐₓ is about 0.1 ng/mL, about 0.5 ng/mL, about 1 ng/mL, about 5 ng/mL, or about 10 ng/mL, including any values or ranges therebetween, following administration of the composition.

In embodiments, the compositions of the present disclosure comprise about 1 mg to about 50 mg of DMT or a pharmaceutically acceptable salt thereof, wherein the administration provides a DMT Tₘₐₓ of about 0.3 h to about 2 h and a DMT AUCₗₐₛₜ of about 0.1 ng·h/mL to about 10 ng h/mL following administration. In embodiments, the DMT Tₘₐₓ is about 0.3 h, about 0.4 h, about 0.5 h, about 0.6 h, about 0.7 h, about 0.8 h, about 0.9 h, about 1.0 h, about 1.2 h, about 1.4 h, about 1.6 h, about 1.8 h, about 2.0 h, about 2.2 h, about 2.4 h, about 2.6 h, about 2.8 h, about 3.0 h, about 3.2 h, about 3.4 h, about 3.6 h, about 3.8 h, or about 4.0 h, including any values or ranges therebetween following administration of the composition. In embodiments, the DMT AUCₗₐₛₜ is about 0.1 ng·h/mL, about 0.5 ng·h/mL, about 1 ng·h/mL, about 5 ng·h/mL, or about 10 ng·h/mL, including any values or ranges therebetween, following administration of the composition.

### Methods of Use

In aspects, the present disclosure provide methods of treatment of a disease or disorder, wherein the method comprises administering a therapeutically effective amount of the composition of the present disclosure to a patient in need thereof. The disease or disorder is neurological disease, disorder, or condition, for example, a neuropsychiatric disorder.

As referred to herein, a “neurological disease, disorder, or condition” refers to a disease, disorder, or condition comprising: a neuropsychiatric disorder, such as depressive disorder or depression (including severe depression such as treatment-resistant depression (TRD), major depressive disorder (MDD) and persistent depressive disorder), alexithymia, catatonic depression, a depressive disorder due to a medical condition, postpartum depression, premenstrual dysphoric disorder, seasonal affective disorder, anxiety, anxiety disorder, social anxiety disorder (SAD), general anxiety disorder (GAD), avolition disorder, bipolar disorder (including bipolar I disorder and bipolar II disorder), post-traumatic stress disorder (PTSD), body dysmorphic disorder, abnormalities of mood or emotion, including the above conditions, dysthymia, schizoaffective disorder, schizophrenia and other psychotic disorders, panic disorder, traumatic stress disorders, phobic disorders, and personality disorders with abnormal mood, such as borderline personality disorder, schizoid and schizotypal disorders, suicide ideation, rumination/unproductive repetitive thoughts negatively impacting one's behavior, mood, and/or ability to focus, obsessive-compulsive disorder, addiction (including substance use disorders, such as addiction to nicotine, alcohol, cocaine, opioids, amphetamine, methamphetamine, heroin, morphine, phencyclidine, 3,4-methylenedioxy-methamphetamine, as well as other addictive substances), addictive behavior (including eating, gambling, sex, pornography, videogames, work, exercise, spiritual obsession, self-harm, travel and shopping addiction, etc.), eating disorders (including anorexia nervosa, bulimia nervosa and binge eating disorder), pain (including pain associated with migraine or headache or chronic pain), prolonged grief disorder, paranoid personality disorder, Autoimmune OCD (adult), behavioral symptoms/anxiety in Fragile X (adult), or Xenomelia.

In embodiments, the present disclosure provide methods of treating treatment-resistant depression (TRD) in a patient in need thereof. In embodiments, a patient with treatment-resistant depression has one treatment attempt, two treatment attempts, three treatment attempts, four treatment attempts, or five treatment attempts. In embodiments, patients with treatment-resistant depression have been diagnosed with major depressive disorder (MDD) and have been unable to respond to three or more treatment attempts. In embodiments, patients with treatment-resistant depression have been diagnosed with bipolar disorder and have been unable to respond to a single treatment attempt. In embodiments, a patient to be treated has not responded to an adequate dose and duration of at least two and no more than five antidepressant medications for the current depressive episode.

In embodiments, the disorder can be diagnosed in accordance with the Diagnostic and Statistical Manual of Mental Disorders-Fifth Edition (DSM-5) published by the American Psychiatric Association. In embodiments, the patient has moderate or severe major depressive disorder as indicated by a Montgomery-Åsberg Depression Rating Scale (MADRS) total score of 20 or more or by a Hamilton Depression Rating Scale (HAM-D) score of 17 or more. In embodiments, the patient has severe major depressive disorder as indicated by a MADRS total score of 35 or more or by a HAM-D score of 25 or more. In embodiments, the patient has a treatment-resistant form of major depressive disorder as indicated by a MADRS score of about 20 or more. In embodiments, the patient that is treated with the methods of the present disclosure has a MADRS score that is at least about 20, at least about 25, at least about 30, at least about 35, at least about 40, at least about 45, at least about 50, at least about 55, about 20-about 25, about 25-about 30, about 30-about 35, about 35-about 40, about 40-about 45, about 45-about 50, about 50-about 55, about 55-about 60, about 25-about 35, about 35-about 45, about 45-about 60, about 25-about 40, or about 40-about 60.

Measures of treatment effect by the methods of the present disclosure include, but are not limited to: Montgomery-Asberg Depression Rating Scale (MADRS), Quality of Life Enjoyment and Satisfaction Questionnaire Short Form, Range of Impaired Functioning Tool, Sheehan Disability Scale, Patient Rated Inventory of Side Effects (PRISE), Columbia-Suicide Severity Rating Scale (C-SSRS), Quick Inventory of Depressive Symptomatology, Self Report (QIDS-SR), Clinical Global Impression (CGI) scale, Massachusetts General Hospital Cognitive and Physical Functioning Questionnaire (CPFQ), 17-item Hamilton Rating Scale for Depression (HAM-D17), Massachusetts General Hospital Antidepressant Treatment Response Questionnaire (MGH ATRQ), 16-item Quick Inventory of Depressive Symptomatology-Self Report (QIDS-SR16), Sheehan Disability Scale (SDS), Clinical Global Impression of Severity of Illness (CGI-S), Clinical Global Impression of Change (CGI-C), EuroQOL 5 Dimension 5 Level (EQ-5D-5L), Patient Global Impression of Change (PGIC), 7-item Generalized Anxiety Disorder (GAD-7), Clinical Global Impressions-Improvement (CGI-I). Sheehan Disability Scale (SDS), 16-item Quick Inventory of Depressive Symptomatology-Self Report (QIDS-SR16), Hamilton Anxiety Scale (HAM-A), Massachusetts General Hospital Cognitive and Physical Functioning Questionnaire (CPFQ), CPFQ-Cognitive subscales (Items 4 to 7), Brief Psychiatric Rating Scale (BPRS), Digit Symbol Substitution Test (DSST), Rey Auditory Verbal Learning Task (RAVLT), Trail Making Test (TMT), Stroop Colour Naming Test (STROOP), Simple Reaction Time (SRT), Choice Reaction Time (CRT). Other procedures, methodologies, or techniques useful for assessing treatment effects known to those skilled in the art may alternatively be used.

In embodiments, the methods of the present disclosure provides a reduction in the patient's MADRS total score by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100% or more, compared to prior to administration. In embodiments, the methods of the present disclosure provides a reduction in the patient's MADRS total score by at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, or more, compared to prior to administration. In embodiments, the methods of the present disclosure provides remission as assessed by patient's MADRS total score equal to or less than 10.

In embodiments, the methods of the present disclosure provide a reduction in the patient's HAM-D score by at least about 50%, at least about 55%, at least about 60%, at least about 65%, at least about 70%, at least about 75%, at least about 80%, at least about 85%, at least about 90%, at least about 95%, at least about 100% or more, compared to prior to administration. In embodiments, the methods of the present disclosure provide a reduction in the patient's HAM-D score by at least about 5, at least about 6, at least about 7, at least about 8, at least about 9, at least about 10, at least about 11, at least about 12, at least about 13, at least about 14, at least about 15, or more, compared to prior to administration. In embodiments, the methods of the present disclosure provides remission as assessed by patient's HAM-D score equal to or less than 7.

In embodiments, the clinical response, as assessed by at least 50% improvement of the MADRS or HAM-D score, compared to the respective score prior to administration, persists until at least about 6 days, at least about 14 days, at least about 28 days, after the last administration of the composition described herein.

Depression may be manifested by depressive symptoms. These symptoms may include psychological changes such as changes in mood, feelings of intense sadness, despair, mental slowing, loss of concentration, pessimistic worry, agitation, anxiety, irritability, guilt, anger, feelings of worthlessness, reckless behavior, suicidal thoughts or attempts, and/or self-deprecation. Physical symptoms of depression may include insomnia, anorexia, appetite loss, weight loss, weight gain, decreased energy and libido, fatigue, restlessness, aches, pains, headaches, cramps, digestive issues, and/or abnormal hormonal circadian rhythms.

In embodiments, the methods provided herein reduce at least one sign or symptom of depressive disorder.

In embodiments, the methods provided herein reduce at least one sign or symptom of major depressive disorder.

### NUMBERED EMBODIMENTS OF THE DISCLOSURE

In addition to the disclosure above, the Examples below, and the appended claims, the disclosure sets forth the following numbered embodiments.

1. A transmucosal composition comprising:
(a) an active layer comprising N,N-dimethyltryptamine (DMT) or a pharmaceutically acceptable salt or prodrug thereof in a polymeric carrier matrix, and
(b) a backing layer, wherein the active layer comprises about 0.5 wt % to about 20 wt % of DMT or a pharmaceutically acceptable salt or prodrug thereof, and wherein the active layer comprises a plasticizer.

2. The composition of embodiment 1, wherein the composition comprises about 70 wt % to about 90 wt % of the active layer.

3. The composition of embodiment 1, wherein the composition comprises about 10 wt % to about 30 wt % of the backing layer.

4. The composition of embodiment 1, wherein the DMT or a pharmaceutically acceptable salt or prodrug thereof is amorphous.

5. The composition of embodiment 1, wherein the DMT or a pharmaceutically acceptable salt or prodrug thereof is amorphous as determined by:
(a) a differential scanning calorimetry (DSC) thermogram lacking a sharp melting endotherm of crystalline DMT and/or lacking an indication of phase change, or
(b) a powder x-ray diffractogram free of any discernable peaks.

6. The composition of any one of embodiments 1-5, wherein the DMT is a DMT free base.

7. The composition of any one of embodiments 1-6, wherein the active layer comprises about 1 wt %, about 2 wt %, about 3 wt %, about 5 wt %, about 7 wt %, about 9 wt %, about 12 wt %, about 15 wt %, about 18 wt %, or about 20 wt % of the DMT or a pharmaceutically acceptable salt or prodrug thereof.

8. The composition of embodiment 7, wherein the active layer comprises about 1 mg to about 30 mg of the DMT or an equivalent amount of the pharmaceutically acceptable salt or prodrug thereof.

9. The composition of embodiment 1, wherein the active layer comprises about 1 mg to about 5 mg, about 5 mg to about 15 mg, about 15 mg to about 20 mg, about 20 mg to about 30 mg of the DMT or an equivalent amount of the pharmaceutically acceptable salt or prodrug thereof.

10. The composition of embodiment 1, wherein the composition comprises about 5 mg, about 15 mg, about 20 mg, about 30 mg of the DMT or an equivalent amount of the pharmaceutically acceptable salt or prodrug thereof.

11. The composition of embodiment 1, wherein the polymeric carrier matrix comprises a mucoadhesive polymer.

12. The composition of any one of embodiments 1-11, wherein the polymeric carrier matrix comprises a cellulose derivative, a polyacrylic acid, a polyacrylate, a polyethylene oxide, polyvinyl pyrrolidone, poly(vinyl pyrrolidone-co-vinyl acetate), hydroxyethyl cellulose, hydroxypropyl cellulose (HPC), copovidone, sodium carboxymethylcellulose, hydroxypropylmethyl cellulose, polyvinyl alcohol, propylene glycol alginate ester, tragacanth, alginate, a gum, a soluble starch, gelatin, lectin, pectin, chitosan, or any combination thereof.

13. The composition of embodiment 12, wherein the polymeric carrier matrix comprises: (a) HPC having an average formula weight of about 140 KDa, wherein a 2 wt % aqueous solution of the HPC provides a solution viscosity of about 6 to about 10 mPa·S, and (b) HPC having an average formula weight of about 370 KDa, wherein a 2 wt % aqueous solution of the HPC provides a solution viscosity of about 150 to about 400 mPa·S.

14. The composition of embodiment 1, wherein the polymeric carrier matrix stabilizes the DMT in an amorphous form within the transmucosal composition.

15. The composition of any one of embodiments 1-14, wherein the active layer comprises about 70 wt % to about 90 wt % of the polymeric carrier matrix.

16. The composition of embodiment 1, wherein the active layer comprises about 10 wt % to 20 wt % of the plasticizer.

17. The composition of embodiment 16, wherein the plasticizer is polyethylene glycol (PEG), propylene glycol, glycerol, triacetin, castor oil, or any combination thereof.

18. The composition of any one of embodiments 1-17, wherein the active layer comprises an anti-foaming agent.

19 The composition of embodiment 18, wherein the active layer comprises about 0.05 wt % to about 1 wt % of the anti-foaming agent.

20 The composition of embodiment 18 or 19, wherein the anti-foaming agent is silicone, polyglycol, or any combination thereof.

21 The composition of embodiment 20, wherein the anti-foaming agent is simethicone.

22. The composition of any one of embodiments 1-21, wherein the active layer comprises a buffering agent.

23. The composition of embodiment 22, wherein the buffering agent comprises citric acid, tartaric acid, fumaric acid, succinic acid, malic acid, sodium citrate, sodium tartrate, sodium succinate, sodium fumarate, sodium hydroxide, or any combination thereof.

24. The composition of embodiment 22 or 23, wherein the active layer comprises about 0.1 wt % to about 1 wt % of the buffering agent.

25 The composition of any one of embodiments 1-24, wherein the active layer comprises a permeation enhancer.

26 The composition of embodiment 25, wherein the permeation enhancer comprises d-α-tocopheryl polyethylene glycol succinate (Vitamin E TPGS), a bile salt, cetylpyridinium chloride (CPC), sodium lauryl sulfate (SLS), Tween 80, L-menthol, oleic alcohol, oleic acid, oleyl oleate, levulinic acid, propylene glycol, dipropylene glycol, ethanol, or any combination thereof.

27 The composition of embodiment 26, wherein the permeation enhancer comprise Vitamin E TPGS.

28 The composition of any one of embodiments 25-27, wherein the active layer comprises about 0.1 wt % to about 1 wt % of the permeation enhancer.

29. The composition of any one of embodiments 1-28, wherein the active layer comprises an antioxidant.

30 The composition of embodiment 29, wherein the antioxidant comprises α-tocopherol, tocopherol acetate, L-Glutathione, L-cysteine, ascorbic acid, ascorbyl palmitate, propyl gallate, butylated hydroxytoluene (BHT), butylated hydroxyanisole (BHA), Tocobiol, Ethylenediaminetetraacetic acid (EDTA), or any combination thereof.

31 The composition of embodiment 29 or 30, wherein the active layer comprises at least about 2 wt % of the antioxidant.

32. The composition of any one of embodiments 25-31, wherein the active layer comprises about 2 wt % to about 10 wt %, about 4 wt % to about 8 wt %, or about 5 wt % to about 6 wt % of the permeation enhancer, and the antioxidant.

33. The composition of any one of the preceding embodiments, wherein the active layer comprises a sweetening agent and/or a flavoring agent.

34 The composition of embodiment 33, wherein the active layer comprises about 0.01 wt % to about 1 wt % of the sweetening agent and/or the flavoring agent.

35. The composition of embodiment 33 or 34, wherein the sweetening agent comprises advantame, sucrose, dextrose, fructose, glucose, maltose, saccharin, sucralose, neotame, cyclamate, aspartame, acesulfame-K, or any combination thereof.

36. The composition of embodiment 35, wherein the sweetening agent comprises advantame.

37. The composition of any one of embodiments 33-36, wherein the flavoring agent comprises lime flavor, lemon flavor, peppermint oil, cinnamon oil, vanilla extract, menthol, L-menthol, or any combination thereof.

38. The composition of any one of the preceding embodiments, wherein the active layer comprises a coloring agent.

39 The composition of embodiment 38, wherein the active layer comprises about 0.1 wt % to about 1.5 wt % of the coloring agent.

40 The composition of embodiment 38 or 39, wherein the coloring agent is selected from D&C or FD&C red, yellow, green, blue, iron oxide red, iron oxide yellow, titanium dioxide, and any combination thereof.

41 The composition of any of the preceding embodiments, wherein the active layer comprises
(a) about 1 wt % to about 20 wt % of DMT;
(b) about 10 wt % to about 20 wt % of the plasticizer;
(c) about 70 wt % to about 90 wt % of polymeric carrier matrix;
(d) about 0.05 wt % to 1 wt % of the anti-foaming agent;
(e) about 0.01 wt % to about 1 wt % of the sweetening agent;
(f) about 0.1 wt % to about 1.5 wt % of the coloring agent;
(g) about 0.1 wt % to about 1 wt % of the buffering agent;
(h) about 0.1 wt % to about 1 wt % of the permeation enhancer; and
(i) about 2 wt % to about 6 wt % of the antioxidant.

42 The composition of any of the preceding embodiments, wherein the active layer comprises
(a) about 2 wt % to about 3 wt % of the DMT;
(b) about 10 wt % to about 20 wt % of PEG300 and triacetin;
(c) about 0.05 wt % to 1 wt % of the anti-foaming agent; and
(d) at least about 70 wt % of the polymeric carrier matrix comprising:
   (i) Copovidone,
   (ii) HPC having an average formula weight of about 140 KDa, wherein a 2 wt % aqueous solution of the HPC provides a solution viscosity of about 6 to about 10 mPa·S,
   (iii) HPC having an average formula weight of about 370 KDa, wherein a 2 wt % aqueous solution of the HPC provides a solution viscosity of about 150 to about 400 mPa·S, and
   (iv) Sodium carboxymethylcellulose.

43. The composition of any of the preceding embodiments, wherein the active layer comprises
(a) about 6 wt % to about 9 wt % of the DMT;
(b) about 10 wt % to about 20 wt % of PEG300 and triacetin;
(c) about 0.05 wt % to 1 wt % of the anti-foaming agent; and
(d) at least about 70 wt % of the polymeric carrier matrix comprising:
   (i) Copovidone,
   (ii) HPC having an average formula weight of about 140 KDa, wherein a 2 wt % aqueous solution of the HPC provides a solution viscosity of about 6 to about 10 mPa·S,
   (iii) HPC having an average formula weight of about 370 KDa, wherein a 2 wt % aqueous solution of the HPC provides a solution viscosity of about 150 to about 400 mPa·S, and
   (iv) Sodium carboxymethylcellulose.

44. The composition of any one of the preceding embodiments, wherein the active layer has a pH of about 7.5 to about 9.

45 The composition of any one of the preceding embodiments, wherein the active layer has a pH of about 8 to about 9.

46. The composition of any one of the preceding embodiments, wherein the active layer has a pH of about 8.5 to about 9.

47 The composition of any one of the preceding embodiments, wherein the active layer has a pH of about 8.6.

48. The composition of any of the preceding embodiments, wherein the active layer has a thickness of about 0.01 mm to about 1.5 mm.

49. The composition of any one of the preceding embodiments, wherein the active layer has a thickness of about 0.5 mm to about 1.5 mm.

50 The composition of embodiment 48 or 49, wherein the active layer comprises about 0.5 mg/cm² to about 5 mg/cm² the DMT or the pharmaceutically acceptable salt or prodrug thereof.

51. The composition of any one of the preceding embodiments, wherein the backing layer reduces or prevents the DMT from being transported across the backing layer while the DMT or the pharmaceutically acceptable salt or prodrug thereof is being administered.

52 The composition of any one of the preceding embodiments, wherein the backing layer comprises a polymeric matrix.

53. The composition of embodiment 52, wherein the polymeric matrix comprises, hydroxypropyl cellulose (HPC), sodium carboxymethylcellulose, polyethylene glycol, and copovidone.

54. The composition of embodiment 52 or 53, wherein the backing layer comprises 60 wt % to about 90 wt % of the polymeric matrix

55. The composition of any one of the preceding embodiments, wherein the backing layer comprises a coloring agent.

56. The composition of embodiment 55, wherein the coloring agent comprises titanium dioxide.

57. The composition of embodiment 55 or 56, wherein the backing layer comprises about 1 wt % to about 10 wt % of the coloring agent.

58. The composition of any of the preceding embodiments, wherein the backing layer comprises sodium hydroxide.

59. The composition of embodiment 58, wherein the backing layer comprises about 0.1 wt % to about 5 wt % sodium hydroxide.

60. The composition of any of the preceding embodiments, wherein the backing layer comprises
(a) about 60 wt % to about 80 wt % of Hydroxypropyl Cellulose;
(b) about 3 wt % to about 10 wt % of Copovidone;
(c) about 5 wt % to about 10 wt % of Sodium Carboxymethylcellulose;
(d) about 10 wt % to about 20 wt % of Polyethylene Glycol;
(e) about 1 wt % to about 3 wt % of Titanium dioxide; and
(f) about 0.5 wt % to about 2 wt % of Sodium Hydroxide NF solution 50% (w/w).

61. The composition of any of the preceding embodiments, the composition comprising:
an active layer comprising
(a) about 1 wt % to about 20 wt % of the DMT;
(b) about 10 wt % to about 20 wt % of PEG300 and triacetin;
(c) at least about 70 wt % of the polymeric carrier matrix comprising:
   (i) about 20 wt % to about 40 wt % of Copovidone,
   (ii) about 20 wt % to about 40 wt % of HPC having an average formula weight of about 140 KDa, wherein a 2 wt % aqueous solution of the HPC provides a solution viscosity of about 6 to about 10 mPa·S,
   (iii) about 1 wt % to about 10 wt % of HPC having an average formula weight of about 370 KDa, wherein a 2 wt % aqueous solution of the HPC provides a solution viscosity of about 150 to about 400 mPa·S; and
   (iv) about 1 wt % to about 5 wt % of Sodium carboxymethylcellulose;
(d) about 0.05 wt % to about 1 wt % of simethicone;
(e) about 1 wt % to about 10 wt % of L-Glutathione;
(f) about 0.1 wt % to about 1 wt % of Sodium hydroxide;
(g) about 0.01 wt % to about 0.1 wt % of Advantame;
(h) about 0.1 wt % to about 1 wt % of Sucralose;
(i) about 0.1 wt % to about 1.5 wt % of FD&C Yellow 6 powder, D&C Yellow #10 Aluminum Lake, FD&C Blue #1 Aluminum Lake, and Titanium dioxide;
(j) about 0.1 wt % to about 1 wt % of Arome Type Suppresseur Amertume;
(k) about 0.1 wt % to about 1 wt % of Evospray Natural Lime Flavor;
(l) about 0.1 wt % to about 1 wt % of Vitamin E TPGS; and
a backing layer comprising:
(a) about 3 wt % to about 10 wt % of Copovidone;
(b) about 60 wt % to about 80 wt % of Sodium Carboxymethylcellulose;
(c) about 10 wt % to about 20 wt % of Polyethylene Glycol;
(d) about 1 wt % to about 3 wt % of Titanium Dioxide; and
(e) about 0.5 wt % to about 2 wt % of Sodium Hydroxide solution 50% (W/W).

62. The composition of any of the preceding embodiments, wherein the composition is suitable for transmucosal application.

63 The composition of any one of embodiments 1-62, wherein the composition is suitable for buccal, sublingual, gingival, on the tongue, nasal, vaginal, or a rectal application.

64. The composition of any of the preceding embodiments, wherein the administration of the composition to the oral mucosa of a patient in need thereof provides a DMT Tₘₐₓ of about 0.3 h to about 2 h and a DMT Cₘₐₓ of about 0.1 ng/ml to about 10 ng/mL following administration.

65. The composition of any of the preceding embodiments, wherein the administration of the composition to the oral mucosa of a patient in need thereof provides a DMT Tₘₐₓ of about 0.3 h to about 2 h and a DMT AUCₗₐₛₜ of about 0.1 ng h/mL to about 10 ng h/mL following administration.

66. A method of treating treatment resistant depression in a patient in need thereof, the method comprising administering the transmucosal composition of any one of embodiments 1-62.

67. The method of embodiment 66, wherein the administration provides a DMT Tₘₐₓ of about 0.3 h to about 2 h and a DMT Cₘₐₓ of about 0.1 ng/ml to about 10 ng/ml following administration.

68 The method of embodiment 67, wherein the administration provides a DMT Tₘₐₓ of about 0.3 h to about 2 h and a DMT AUCₗₐₛₜ of about 0.1 ng·h/mL to about 10 ng h/mL following administration.

69. The method of any one of embodiments 66-68, wherein the composition is administered buccally.

70 The method of any one of embodiments 66-69, wherein the composition is administered through a transmucosal application.

71. The method of any one of embodiments 66-70, wherein the composition is administered once about every about 2 weeks to about 6 months.

72. The method of any one of embodiments 66-71, wherein the administration provides a reduction in the patient's Montgomery-Asberg Depression Rating Scale (MADRS) total score by at least about 20%, at least about 30%, at least about 40%, or at least about 50% compared to prior to the administration.

73. The method of any one of embodiments 66-72, wherein the administration provides a reduction in the patient's MADRS total score by at least about 6 compared to prior to the administration.

74 The method of any one of embodiments 66-73, wherein the administration provides remission according to the patient's MADRS total score after the administration.

75 The method of any one of embodiments 66-74, wherein the patient is at least 18 years old.

76 The method of any one of embodiments 66-75, wherein the patient has a diagnosis of moderate or severe major depressive disorder (MDD) without psychotic features and is currently experiencing a major depressive episode.

77. The method of embodiment 76, wherein the patient has the onset of the first episode of MDD before age 55.

78 The method of embodiment 76 or 77, wherein the patient has not responded to an adequate dose and duration of at least two and no more than five antidepressant medications for the current depressive episode.

79 The method of any one of embodiments 76-78, wherein the patient has a MADRS total score of at least about 20 prior to the administration.

### EXAMPLES

The following examples are provided to further illustrate the embodiments of the present disclosure but are not intended to limit the scope of the claims. While they are typical of those that might be used, other procedures, methodologies, or techniques known to those skilled in the art may alternatively be used.

### Example 1. DMT Low Dose Oral Transmucosal Film (OTF)

The formulations of DMT oral transmucosal films (OTF, also known as buccal film) in wet compositions and dried compositions are provided in Table 10 and Table 11, respectively.

TABLE 10
% Wet Composition of DMT formulations

| | | | | | | % Wet |
|---|---|---|---|---|---|---|
| | | | % Wet | % Wet | % Wet | Composition |
| | | | Composition | Composition | Composition | Backing |
| | Quality | | 60 mg | 5 mg | 15 mg | (5 mg & 15 mg |
| Component | Reference | Function | Active | Active | Active | & 60 mg) |
| DMT | In-house | Active | 14.73 | 1.49 | 4.48 | — |
| Hydroxypropyl cellulose | USP | Polymer | 0.47 | 2.65 | 2.65 | — |
| (Klucel GXF) | | (Film | | | | |
| | | former) | | | | |
| Hydroxypropyl cellulose | USP | Polymer | 2.34 | 13.42 | 13.20 | 15.99 |
| (Nisso HPC-L) | | (Film | | | | |
| | | former) | | | | |
| Hydroxypropyl cellulose | USP | Polymer | 9.49 | — | — | — |
| (Nisso HPC-SSL) | | (Film | | | | |
| | | former) | | | | |
| Copovidone (Plasdone | USP | Polymer | 1.07 | 22.30 | 18.94 | 1.39 |
| S-630) | | (Film | | | | |
| | | former) | | | | |
| Sodium | USP | Polymer | 1.40 | — | — | 1.82 |
| carboxymethylcellulose | | (Film | | | | |
| | | former) | | | | |
| Sodium hydroxide | NF | pH modifier | — | 0.31 | 0.18 | 0.17 |
| Citric acid | USP | pH modifier | 2.07 | — | — | — |
| Simethicone | USP | Anti- | — | 0.09 | 0.19 | — |
| | | foaming | | | | |
| | | agent | | | | |
| L-Glutathione | EP | Antioxidant | 0.87 | 2.24 | 2.04 | — |
| Vitamin E TPGS | NF | Permeation | 1.47 | 0.15 | 0.45 | — |
| | | enhancer | | | | |
| Maltitol | NF | Disintegrating | 4.58 | — | — | — |
| | | agent | | | | |
| Polyethylene Glycol 300 | NF | Plasticizer | — | 4.05 | 4.64 | 3.35 |
| (PEG-300) | | | | | | |
| Triacetin | USP | Plasticizer | — | 2.37 | 1.89 | — |
| Advantame | FCC | Sweetener | 0.05 | 0.005 | 0.015 | — |
| Sucralose | USP | Sweetener | 0.62 | 0.06 | 0.19 | — |
| Arome type suppresseur | FCC | Flavor | 1.28 | 0.13 | 0.39 | — |
| amertume | | | | | | |
| Evospray natural lime | FCC | Flavor | 0.66 | 0.07 | 0.20 | — |
| flavor | | | | | | |
| FD&C Yellow# 6 | FD&C | Colorant | 0.005 | 0.01 | 0.01 | — |
| powder | | | | | | |
| D&C Yellow# 10 | D&C | Colorant | — | 0.02 | 0.02 | — |
| Aluminum Lake | | | | | | |
| FD&C Blue# 1 | FD&C | Colorant | — | 0.004 | 0.004 | — |
| Aluminum Lake | | | | | | |
| Titanium dioxide | USP | Colorant | — | 0.27 | 0.27 | 0.49 |
| Methyl alcohol | USP | Solvent | 23.57 | 5.04 | 5.02 | 30.65 |
| Purified water | USP | Solvent | 35.35 | 45.32 | 45.22 | 46.14 |

| | | | | |
|---|---|---|---|---|
| Total | 100.0 | 100.0 | 100.0 | 100.0 |

TABLE 11
% Dry Composition of DMT formulations

| | | | % Dry | % Dry | % Dry |
|---|---|---|---|---|---|
| | | | Composition | Composition | Composition |
| | | | 60 mg | 5 mg | 15 mg |
| Component | Quality Reference | Function | Bilayer | Bilayer | Bilayer |
| DMT | In-house | Active | 29.03 | 2.35 | 7.03 |
| Hydroxypropyl | USP | Polymer | 0.92 | 4.17 | 4.16 |
| cellulose (Klucel GXF) | | (Film former) | | | |
| Hydroxypropyl | USP | Polymer | 17.70 | 36.21 | 35.79 |
| cellulose (Nisso HPC-L) | | (Film former) | | | |
| Hydroxypropyl | USP | Polymer | 18.71 | — | — |
| cellulose (Nisso HPC- | | (Film former) | | | |
| SSL) | | | | | |
| Copovidone (Plasdone | USP | Polymer | 3.25 | 36.39 | 31.06 |
| S-630) | | (Film former) | | | |
| Sodium | USP | Polymer | 4.26 | 1.72 | 1.72 |
| carboxymethylcellulose | | (Film former) | | | |
| Sodium hydroxide | NF | pH modifier | 0.14 | 0.65 | 0.44 |
| Citric acid | USP | pH modifier | 4.08 | — | — |
| Simethicone | USP | Anti-foaming | — | 0.15 | 0.30 |
| | | agent | | | |
| L-Glutathione | EP | Antioxidant | 1.71 | 3.52 | 3.21 |
| Vitamin E TPGS | NF | Permeation | 2.90 | 0.23 | 0.70 |
| | | enhancer | | | |
| Maltitol | NF | Disintegrating | 9.03 | — | — |
| | | agent | | | |
| Polyethylene Glycol | NF | Plasticizer | 2.74 | 9.53 | 10.44 |
| 300 (PEG-300) | | | | | |
| Triacetin | USP | Plasticizer | — | 3.73 | 2.97 |
| Advantame | FCC | Sweetener | 0.10 | 0.01 | 0.02 |
| Sucralose | USP | Sweetener | 1.22 | 0.10 | 0.29 |
| Arome type | FCC | Flavor | 2.52 | 0.20 | 0.61 |
| suppresseur amertume | | | | | |
| Evospray natural lime | FCC | Flavor | 1.29 | 0.10 | 0.31 |
| flavor | | | | | |
| FD&C Yellow# 6 | FD&C | Colorant | 0.01 | 0.02 | 0.02 |
| powder | | | | | |
| D&C Yellow# 10 | D&C | Colorant | — | 0.03 | 0.03 |
| Aluminum Lake | | | | | |
| FD&C Blue# 1 | FD&C | Colorant | — | 0.01 | 0.01 |
| Aluminum Lake | | | | | |
| Titanium dioxide | USP | Colorant | 0.40 | 0.88 | 0.87 |
| Methyl alcohol | USP | Solvent | — | — | — |
| Purified water | USP | Solvent | — | — | — |

| | | | |
|---|---|---|---|
| Total | 100.0 | 100.0 | 100.0 |

As DMT acts as a plasticizer in high dose DMT buccal films (e.g., 60 mg and 80 mg films), additional plasticizers (e.g., PEG-300 and Triacetin) are included in low dose DMT films (e.g., 5 mg and 15 mg film) to retain similar physical properties to the high dose DMT buccal films.

The film endurance performance was determined using Mecmesin MultiTest-dV—Motorised Test. The low dose buccal films retain similar film appearance and physical properties (e.g., colour, softness, film weight/thickness), film performance characteristics (e.g., pH, residence time (in buccal), dissolution profile and permeation) and film stability (e.g., storage and shelf-life determination, amorphous DMT) to those of the high dose DMT buccal films.

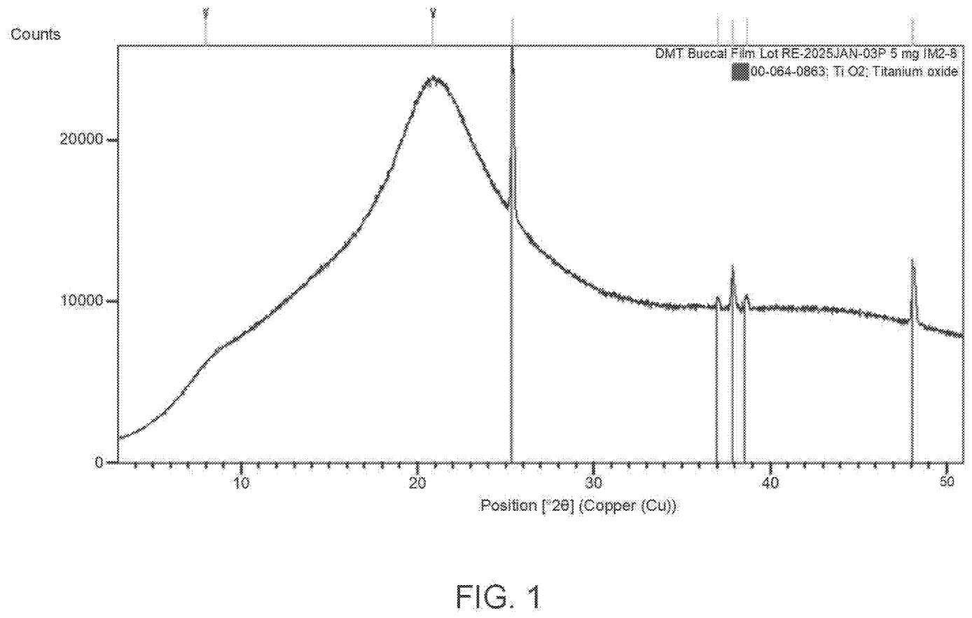

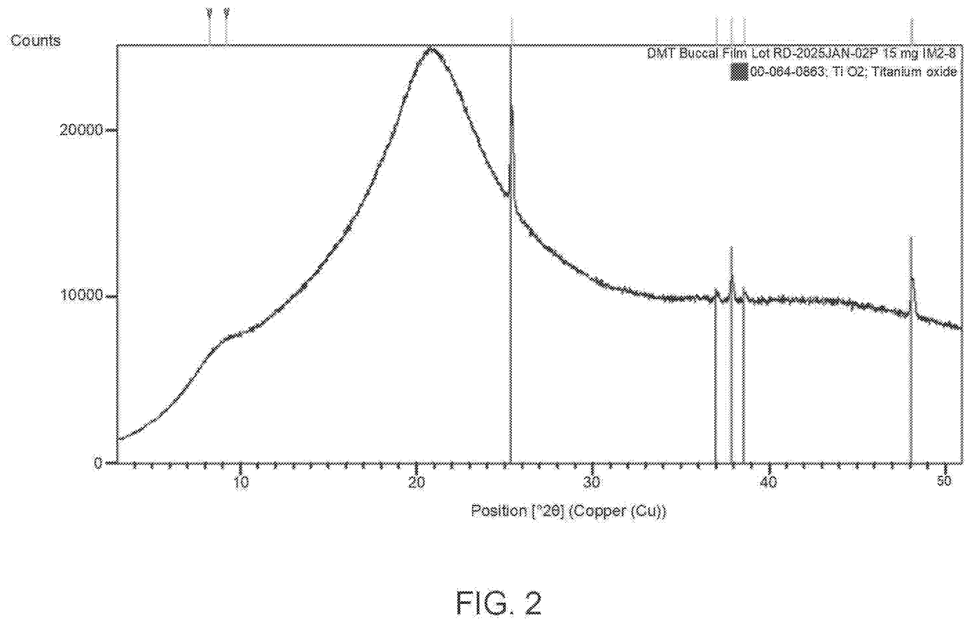

FIGS. 1 and 2 show powder x-ray diffractograms of the 5 mg and 15 mg films, which are free of any discernable peaks attributable to crystalline DMT, confirming that the DMT in these films is in an amorphous form.

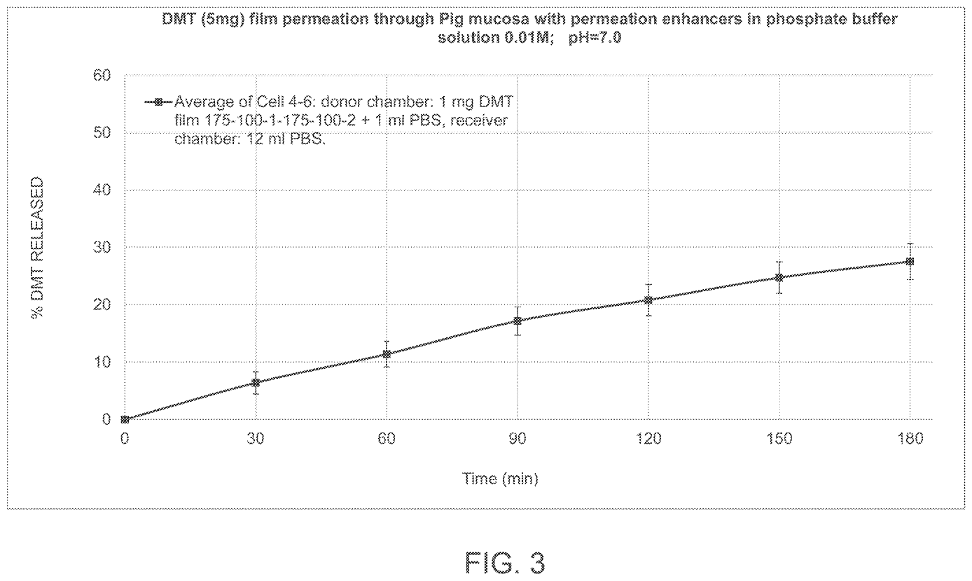

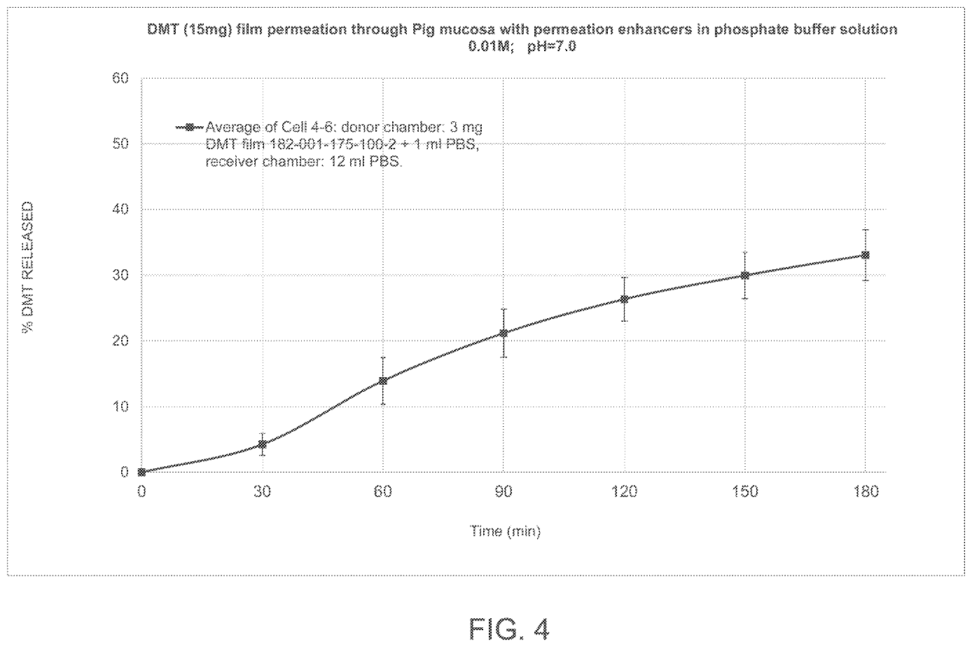

FIGS. 3 and 4 depict the results of ex vivo permeation studies conducted using pig mucosa for the 5 mg and 15 mg DMT films, respectively. The studies were performed in 0.01 M phosphate buffer solution using Vitamin E TPGS as a permeation enhancer. For both the 5 mg and 15 mg films, approximately 30% of the DMT permeated through the mucosa within 180 minutes.

### Example 2 Phase 1 Study of DMT Buccal Film

A phase 1, single-site, double-blind, randomized, placebo-controlled trial to assess pharmacokinetics, pharmacodynamics, safety, and tolerability of ascending doses of DMT buccal film in healthy participants is conducted to evaluate PK/PD, safety, and tolerability of ascending doses of DMT buccal film in healthy participants.

In each cohort, participants will be randomized 3:1 to receive DMT buccal film or matching placebo. A single dose of 5 mg, 10 mg, 15 mg, 20 mg, 25 mg, or 30 mg of DMT in buccal film or placebo will be given via buccal transmucosal administration to participants.

The primary objectives of the study are to assess the plasma pharmacokinetic (PK) characteristics of DMT buccal film and its two predominant metabolites [N,N-dimethyltryptamine-N-oxide (DMT-NO) and indole-3-acetic acid (IAA)]; to characterize dose-proportionality of DMT buccal film; to estimate the inter-participant variability in the single-dose pharmacokinetic (PK) parameters of DMT; and to characterize the subjective effects of DMT buccal film compared to placebo in healthy participants.

The secondary objectives of the study are to assess the safety of DMT buccal film compared to placebo in healthy participants; and to assess the pharmacogenomic profiles following DMT buccal film administration.

The primary endpoints of the study are plasma PK parameters (including but not limited to Cₘₐₓ, Tₘₐₓ, AUC₀₋ₗₐₛₜ) of DMT, DMT-NO, and IAA on Day 1; subjective Intensity Rating Scale (SIRS) score over time: 0.25, 0.50, 0.75, 1.0, 1.5, 2.0, 2.5, 3.5, 4.5, 5.5, 7.5 h postdose on Day 1; maximum SIRS retrospectively assessed at 3.5 hours post-dose on Day 1; challenging Experiences Questionnaire (CEQ) total and subscale scores at 3.5 hours postdose on Day 1; and 5-Dimensional Altered States of Consciousness Rating Scale (5D-ASC) total and subscale scores at 3.5 hours postdose on Day 1.

The plasma PK parameters measured in this study are provided in Table 12.

TABLE 12
Pharmacokinetic Parameters

| Parameter | Definition |
|---|---|
| Cₘₐₓ | Maximum observed plasma concentration. If the same Cₘₐₓ concentration occurs at |
| | different time points, Tₘₐₓ is assigned to the first occurrence of Cₘₐₓ. |
| Tₘₐₓ | Time to maximum concentration. |
| AUCₗₐₛₜ | Area under the drug concentration-time curve, from time zero (time of dosing) to the last |
| | measurable concentration using the ‘Linear Up and Log Down’ method. |
| AUC_{0-inf} | The area under the plasma drug concentration-time curve from time 0 (time of dosing) |
| | extrapolated to infinity. AUC_{0-inf} is calculated as the sum of AUC₀₌ₜ plus the ratio of the |
| | last measurable plasma concentration (Cₗₐₛₜ) to the elimination rate constant (λ_{z}), calculated |
| | by the ‘Linear Up and Log Down’ method. |
| Half-life (t_{1/2}) | Apparent terminal half-life, determined using the following formula: |
| | t_{1/2} = ln(2)/λ_{z} |
| CL/F or CL | Apparent total body clearance, determined using the following formula: |
| | CL/F or CL = Dose in mg/AUC_{0-inf} |
| | (CL for IV infusion and CL/F for buccal route). |
| Vz/F or Vz | Apparent terminal volume of distribution, determined using the following formula: |
| | V_{z}/F or Vz = Dose in mg/λ_{z} × AUC_{0-inf} |
| | (Vz for IV infusion and Vz/F for buccal route). |
| Cₘₐₓ/Dose | Dose-normalized Cₘₐₓ, calculated as Cₘₐₓ divided by the dose in mg administered. |
| AUCₗₐₛₜ/Dose | Dose-normalized AUC0-t, calculated as AUC0-t divided by dose in mg administered. |
| SIRS Max | Maximum SIRS rating over the course of treatment |
| Metabolite Ratio | AUCₗₐₛₜ of the metabolite/AUCₗₐₛₜ of DMT |
| (AUClastₘₑₜ/ | |
| AUClast_{DMT}) | |

Abbreviations: BU = buccal; IV = intravenous; PK = pharmacokinetic.

The pharmacodynamics (PD) parameters measured in this study are provided in Table 13.

TABLE 13
Pharmacodynamics parameters

| Assessment | Description |
|---|---|
| Subjective Intensity | For participants to verbally estimate intensity of the drug effect in real time on a |
| Rating Scale (SIRS) | scale of 0 to 10 (range 0 = ‘No effect/the effects have completely worn off’ to |
| | 10 = ‘Greatest intensity imaginable’). SIRS was collected at the PK sampling |
| | timepoints. |
| Challenging | Assessed unpleasant reactions to psychedelic drugs. It covered self-reporting on |
| Experience | acute affective, cognitive, and somatic symptoms pertaining with subscales of |
| Questionnaire | fear, grief, physical distress, insanity, isolation, death, and paranoia; 26 items on |
| (CEQ) | a self-report Likert scale (range 0 to 5: 0 = ‘None/not at all’ to 5 = ‘Extreme/more |
| | than ever before in my life’). |
| Mystical | The MEQ is designed to measure four forms of mystical experiences that can be |
| Experience | experienced during a psychedelic experience. The measure includes 30 items that |
| Questionnaire | are rated on a self-report Likert scale (range 0 to 5: 0 = “none/not at all” to 5 = |
| (MEQ) | “extreme [more than any other time in my life and stronger than 4]”). |

The secondary endpoints of the study are incidence and severity of adverse events (AEs), adverse events of special interest (AESIs) and serious adverse events (SAEs) (Baseline to end of study [EOS] Visit); incidence of clinically significant abnormalities in physical and oral cavity examinations, taste survey, vital signs, electrocardiogram parameters, and safety laboratory results (Post dose to EOS Visit); and Columbia-Suicide Severity Rating Scale (C-SSRS) score (Baseline to EOS Visit).

### Example 3 Efficacy, Safety, and Tolerability of Repeated Doses of DMT Buccal Transmucosal Composition in a Phase 2 Clinical Study

A Phase 2, multicenter, double-blind, randomized, placebo-controlled trial is conducted to assess the efficacy, safety, and tolerability of repeated doses of DMT buccal film in participants with treatment resistant depression.

The primary objective is to determine the efficacy of DMT buccal film compared with placebo in improving depressive symptoms in participants with treatment-resistant depression (TRD) 4 weeks after initiating treatment. The endpoint is to assess the change from baseline (prior to administration) in Montgomery-Asberg Depression Rating Scale (MADRS) total score. The secondary objectives are: (1) to assess the efficacy of DMT buccal film compared to placebo in improving depressive symptoms in participants with TRD 6-weeks after initiating treatment; (2) to assess the proportion of treatment responders after DMT buccal film U treatment, compared with placebo, 4 weeks after initiating treatment; (3) to assess the proportion of participants achieving remission after DMT buccal film treatment, compared with placebo, 4 weeks after initiating treatment; and (4) to assess the durability of the antidepressant treatment response 12 weeks after receiving two doses of DMT buccal film, compared to placebo. The corresponding endpoints include (1) change from Baseline in MADRS total score at Day 43; (2) Proportion of treatment responders (defined as ≥50% decrease from Baseline in MADRS total score) at Day 29; (3) Proportion of participants meeting criteria for remission (defined as MADRS total score≤10) at Day 29; and (4) change from Baseline in MADRS total score at Day 99 pre-dose.

This Phase 2 trial will enroll approximately 142 participants (up to a maximum of 168) with TRD, randomized 1:1 to receive a total of 2 double-blinded administrations of DMT buccal film or placebo during the placebo-controlled treatment period. All participants will have their symptoms monitored for 12 weeks (placebo-controlled follow-up period) following the second administration of DMT BU or placebo. All participants will then be re-randomized 1:1 to receive one double-blinded administration of DMT buccal film 30 mg, DMT buccal film 20 mg DMT buccal film 15 mg, DMT buccal film 10 mg, or DMT buccal film 5 mg (non-placebo-controlled treatment period).

Participants are eligible to be included in the study only if all the following criteria apply.

### Age

Participant must be at least 18 years of age inclusive at the time of signing the informed consent.

### Type of Participant and Disease Characteristics

Participant has a diagnosis of recurrent moderate or severe MDD without psychotic features or single-episode major depressive disorder (MDD) with duration greater than or equal to 3 months without psychotic features and is currently experiencing a major depressive episode as defined by the Diagnostic and Statistical Manual of Mental Disorders, Fifth Edition Text Revision (DSM-5-TR) and confirmed by the Mini-International Neuropsychiatric Interview (MINI) and SAFER criteria at Screening.

Participant has onset of first episode of MDD occurred before age 55.

Participant has not responded to an adequate dose and duration of at least two and no more than five antidepressant medications for the current depressive episode, as determined by the Massachusetts General Hospital Antidepressant Treatment Response Questionnaire (and confirmed by clinical interview or clinical documentation if available (e.g., pharmacy, hospital, or other health records of previous treatment). Augmentation with an add-on treatment counts as a second treatment, provided it is approved for the adjunctive treatment of MDD in that country.

Participant has a MADRS total score≥20 at Screening and Day −1.

Participants who are not lactating or pregnant as confirmed by a serum pregnancy test at Screening and negative urine pregnancy test before dosing (applies only to participants of childbearing potential).

Participants are excluded from the study if any of the following criteria apply.

Participant has a current or prior DSM-5-TR diagnosis of a schizophrenia spectrum and other psychotic disorder, substance/medication-induced psychotic disorder, bipolar and related disorder, or any disorder with psychotic features (including MDD with psychotic features), as assessed by medical history and a structured clinical interview (version 7.0.2 MINI).

Participant has a current or prior DSM-5-TR diagnosis of a neurocognitive disorder, intellectual disorder, dissociative disorder, disruptive/impulse-control/conduct disorder, autism spectrum disorder level 2 or 3, or cluster A and B personality disorder, as assessed by medical history and a structured clinical interview (version 7.0.2 MINI). Inclusion of patients with a diagnosis of autism spectrum disorder level 1, or prior diagnosis of cluster C personality disorder may be considered at the discretion of the investigator if the participant no longer meets criteria for the condition and current functioning and/or subthreshold symptoms will not interfere with MDD treatment or compliance in the study.

Participant has a current DMS-5-TR diagnosis of posttraumatic stress disorder, acute stress disorder, obsessive-compulsive disorder, anorexia nervosa, bulimia nervosa, or any other comorbid psychiatric condition that dominates the clinical presentation and would interfere with experimental treatment.

Participant has a mild, moderate, or severe substance use disorder (drug, alcohol, or tobacco) within the 6 months before Screening and/or history of moderate or severe substance use disorder (drug or alcohol) within the previous 1 year before Screening.

Participant has suicidal ideation with some intent to act within 6 months before Screening or a history of suicidal behavior within the past 1 year before Screening. Suicide risk should be informed by clinical judgement, scoring 17 or higher on the MINI Suicidal Scale at Screening, or endorsing “Yes” on Item 4 (active suicidal ideation with some intent to act, without specific plan) or Item 5 (active suicidal ideation with specific plan and intent) on the CSSRS at Day −1, or MADRS item 10 “suicidal thoughts” score=6 at Screening or Baseline.

1. A transmucosal composition comprising:
(a) an active layer comprising N,N-dimethyltryptamine (DMT) or a pharmaceutically acceptable salt or prodrug thereof in a polymeric carrier matrix, and
(b) a backing layer,
wherein the active layer comprises about 0.5 wt % to about 20 wt % of DMT or a pharmaceutically acceptable salt or prodrug thereof, and
wherein the active layer comprises a plasticizer.

2. The composition of claim 1, wherein the composition comprises about 70 wt % to about 90 wt % of the active layer.

3. (canceled)

4. The composition of claim 1, wherein the DMT or a pharmaceutically acceptable salt or prodrug thereof is amorphous, as determined by:
(a) a differential scanning calorimetry (DSC) thermogram lacking a sharp melting endotherm of crystalline DMT and/or lacking an indication of phase change, or
(b) a powder x-ray diffractogram free of any discernable peaks.

5. (canceled)

6. The composition of claim 1, wherein the DMT is a DMT free base.

7. The composition of claim 1, wherein the active layer comprises about 1 wt %, about 2 wt %, about 3 wt %, about 5 wt %, about 7 wt %, about 9 wt %, about 12 wt %, about 15 wt %, about 18 wt %, or about 20 wt % of the DMT or a pharmaceutically acceptable salt or prodrug thereof; or
wherein the active layer comprises about 1 mg to about 30 mg of the DMT or an equivalent amount of the pharmaceutically acceptable salt or prodrug thereof.

8. (canceled)

9. (canceled)

10. (canceled)

11. The composition of claim 1, wherein the polymeric carrier matrix comprises a mucoadhesive polymer.

12. The composition of claim 11, wherein the polymeric carrier matrix comprises a cellulose derivative, a polyacrylic acid, a polyacrylate, a polyethylene oxide, polyvinyl pyrrolidone, poly(vinyl pyrrolidone-co-vinyl acetate), hydroxyethyl cellulose, hydroxypropyl cellulose (HPC), copovidone, sodium carboxymethylcellulose, hydroxypropylmethyl cellulose, polyvinyl alcohol, propylene glycol alginate ester, tragacanth, alginate, a gum, a soluble starch, gelatin, lectin, pectin, chitosan, or any combination thereof.

13. The composition of claim 12, wherein the polymeric carrier matrix comprises:
(a) HPC having an average formula weight of about 140 KDa, wherein a 2 wt % aqueous solution of the HPC provides a solution viscosity of about 6 to about 10 mPa·S, and
(b) HPC having an average formula weight of about 370 KDa, wherein a 2 wt % aqueous solution of the HPC provides a solution viscosity of about 150 to about 400 mPa·S.

14. The composition of claim 1, wherein the polymeric carrier matrix stabilizes the DMT in an amorphous form within the transmucosal composition.

15. The composition of claim 1, wherein the active layer comprises about 70 wt % to about 90 wt % of the polymeric carrier matrix.

16. The composition of claim 1, wherein the active layer comprises about 10 wt % to 20 wt % of the plasticizer.

17. The composition of claim 16, wherein the plasticizer is polyethylene glycol (PEG), propylene glycol, glycerol, triacetin, castor oil, or any combination thereof.

18. The composition of claim 1, wherein the active layer comprises an anti-foaming agent, a buffering agent, a permeation enhancer, an antioxidant, a sweetening agent, a flavoring agent, a coloring agent, or a combination thereof.

19. The composition of claim 18, wherein the active layer comprises about 0.05 wt % to about 1 wt % of the anti-foaming agent.

20. The composition of claim 18, wherein the anti-foaming agent is simethicone, silicone, polyglycol, or any combination thereof.

21. (canceled)

22. (canceled)

23. The composition of claim 18, wherein the buffering agent comprises citric acid, tartaric acid, fumaric acid, succinic acid, malic acid, sodium citrate, sodium tartrate, sodium succinate, sodium fumarate, sodium hydroxide, or any combination thereof.

24. The composition of claim 23, wherein the active layer comprises about 0.1 wt % to about 1 wt % of the buffering agent.

25. (canceled)

26. The composition of claim 18, wherein the permeation enhancer comprises d-α-tocopheryl polyethylene glycol succinate (Vitamin E TPGS), a bile salt, cetylpyridinium chloride (CPC), sodium lauryl sulfate (SLS), Tween 80, L-menthol, oleic alcohol, oleic acid, oleyl oleate, levulinic acid, propylene glycol, dipropylene glycol, ethanol, or any combination thereof.

27. (canceled)

28. The composition of claim 18, wherein the active layer comprises about 0.1 wt % to about 1 wt % of the permeation enhancer.

29. (canceled)

30. The composition of claim 18, wherein the antioxidant comprises α-tocopherol, tocopherol acetate, L-Glutathione, L-cysteine, ascorbic acid, ascorbyl palmitate, propyl gallate, butylated hydroxytoluene (BHT), butylated hydroxyanisole (BHA), Tocobiol, Ethylenediaminetetraacetic acid (EDTA), or any combination thereof.

31. The composition of claim 30, wherein the active layer comprises at least about 2 wt % of the antioxidant.

32. The composition of claim 18, wherein the active layer comprises about 2 wt % to about 10 wt %, about 4 wt % to about 8 wt %, or about 5 wt % to about 6 wt % of the permeation enhancer, and the antioxidant.

33. (canceled)

34. The composition of claim 18, wherein the active layer comprises about 0.01 wt % to about 1 wt % of the sweetening agent and/or the flavoring agent.

35. The composition of claim 18, wherein the sweetening agent comprises advantame, sucrose, dextrose, fructose, glucose, maltose, saccharin, sucralose, neotame, cyclamate, aspartame, acesulfame-K, or any combination thereof.

36. (canceled)

37. The composition of claim 18, wherein the flavoring agent comprises lime flavor, lemon flavor, peppermint oil, cinnamon oil, vanilla extract, menthol, L-menthol, or any combination thereof.

38. (canceled)

39. The composition of claim 18, wherein the active layer comprises about 0.1 wt % to about 1.5 wt % of the coloring agent.

40. The composition of claim 18, wherein the coloring agent is selected from D&C or FD&C red, yellow, green, blue, iron oxide red, iron oxide yellow, titanium dioxide, and any combination thereof.

41. The composition of claim 1, wherein the active layer comprises
(a) about 1 wt % to about 20 wt % of DMT;
(b) about 10 wt % to about 20 wt % of the plasticizer;
(c) about 70 wt % to about 90 wt % of polymeric carrier matrix;
(d) about 0.05 wt % to 1 wt % of the anti-foaming agent;
(e) about 0.01 wt % to about 1 wt % of the sweetening agent;
(f) about 0.1 wt % to about 1.5 wt % of the coloring agent;
(g) about 0.1 wt % to about 1 wt % of the buffering agent;
(h) about 0.1 wt % to about 1 wt % of the permeation enhancer; and
(i) about 2 wt % to about 6 wt % of the antioxidant.

42. The composition of claim 1, wherein the active layer comprises
(a) about 2 wt % to about 3 wt % of the DMT;
(b) about 10 wt % to about 20 wt % of PEG300 and triacetin;
(c) about 0.05 wt % to 1 wt % of the anti-foaming agent; and
(d) at least about 70 wt % of the polymeric carrier matrix comprising:
(i) Copovidone,
(ii) HPC having an average formula weight of about 140 KDa, wherein a 2 wt % aqueous solution of the HPC provides a solution viscosity of about 6 to about 10 mPa·S,
(iii) HPC having an average formula weight of about 370 KDa, wherein a 2 wt % aqueous solution of the HPC provides a solution viscosity of about 150 to about 400 mPa·S, and
(iv) Sodium carboxymethylcellulose.

43. The composition of claim 1, wherein the active layer comprises
(a) about 6 wt % to about 9 wt % of the DMT;
(b) about 10 wt % to about 20 wt % of PEG300 and triacetin;
(c) about 0.05 wt % to 1 wt % of the anti-foaming agent; and
(d) at least about 70 wt % of the polymeric carrier matrix comprising:
(i) Copovidone,
(ii) HPC having an average formula weight of about 140 KDa, wherein a 2 wt % aqueous solution of the HPC provides a solution viscosity of about 6 to about 10 mPa·S,
(iii) HPC having an average formula weight of about 370 KDa, wherein a 2 wt % aqueous solution of the HPC provides a solution viscosity of about 150 to about 400 mPa·S, and
(iv) Sodium carboxymethylcellulose.

44. The composition of claim 1, wherein the active layer has a pH of about 7.5 to about 9.

45. (canceled)

46. (canceled)

47. (canceled)

48. The composition of claim 1, wherein the active layer comprises about 0.5 mg/cm² to about 5 mg/cm² the DMT or the pharmaceutically acceptable salt or prodrug thereof.

49. (canceled)

50. (canceled)

51. The composition of claim 1, wherein the backing layer reduces or prevents the DMT from being transported across the backing layer while the DMT or the pharmaceutically acceptable salt or prodrug thereof is being administered.

52. The composition of claim 1, wherein the backing layer comprises a polymeric matrix.

53. The composition of claim 52, wherein the polymeric matrix comprises hydroxypropyl cellulose (HPC), sodium carboxymethylcellulose, polyethylene glycol, and copovidone.

54. The composition of claim 52, wherein the backing layer comprises 60 wt % to about 90 wt % of the polymeric matrix.

55. (canceled)

56. (canceled)

57. (canceled)

58. The composition of claim 1, wherein the backing layer comprises sodium hydroxide.

59. The composition of claim 58, wherein the backing layer comprises about 0.1 wt % to about 5 wt % sodium hydroxide.

60. The composition of claim 1, wherein the backing layer comprises
(a) about 60 wt % to about 80 wt % of Hydroxypropyl Cellulose;
(b) about 3 wt % to about 10 wt % of Copovidone;
(c) about 5 wt % to about 10 wt % of Sodium Carboxymethylcellulose;
(d) about 10 wt % to about 20 wt % of Polyethylene Glycol;
(e) about 1 wt % to about 3 wt % of Titanium dioxide; and
(f) about 0.5 wt % to about 2 wt % of Sodium Hydroxide NF solution 50% (w/w).

61. The composition of claim 1, the composition comprising:
an active layer comprising
(a) about 1 wt % to about 20 wt % of the DMT;
(b) about 10 wt % to about 20 wt % of PEG300 and triacetin;
(c) at least about 70 wt % of the polymeric carrier matrix comprising:
(i) about 20 wt % to about 40 wt % of Copovidone,
(ii) about 20 wt % to about 40 wt % of HPC having an average formula weight of about 140 KDa, wherein a 2 wt % aqueous solution of the HPC provides a solution viscosity of about 6 to about 10 mPa·S,
(iii) about 1 wt % to about 10 wt % of HPC having an average formula weight of about 370 KDa, wherein a 2 wt % aqueous solution of the HPC provides a solution viscosity of about 150 to about 400 mPa·S; and
(iv) about 1 wt % to about 5 wt % of Sodium carboxymethylcellulose;
(d) about 0.05 wt % to about 1 wt % of simethicone;
(e) about 1 wt % to about 10 wt % of L-Glutathione;
(f) about 0.1 wt % to about 1 wt % of Sodium hydroxide;
(g) about 0.01 wt % to about 0.1 wt % of Advantame;
(h) about 0.1 wt % to about 1 wt % of Sucralose;
(i) about 0.1 wt % to about 1.5 wt % of FD&C Yellow 6 powder, D&C Yellow #10 Aluminum Lake, FD&C Blue #1 Aluminum Lake, and Titanium dioxide;
(j) about 0.1 wt % to about 1 wt % of Arome Type Suppresseur Amertume;
(k) about 0.1 wt % to about 1 wt % of Evospray Natural Lime Flavor;
(l) about 0.1 wt % to about 1 wt % of Vitamin E TPGS; and
a backing layer comprising:
(a) about 60 wt % to about 80 wt % of Hydroxypropyl Cellulose;
(b) about 3 wt % to about 10 wt % of Copovidone;
(c) about 5 wt % to about 10 wt % of Sodium Carboxymethylcellulose;
(d) about 10 wt % to about 20 wt % of Polyethylene Glycol;
(e) about 1 wt % to about 3 wt % of Titanium Dioxide; and
(f) about 0.5 wt % to about 2 wt % of Sodium Hydroxide solution 50% (W/W).

62. The composition of claim 1, wherein the composition is suitable for transmucosal, buccal, sublingual, gingival, on the tongue, nasal, vaginal, or a rectal application.

63. (canceled)

64. The composition of claim 1, wherein the administration of the composition to the oral mucosa of a patient in need thereof provides a DMT Tₘₐₓ of about 0.3 h to about 2 h and a DMT Cₘₐₓ of about 0.1 ng/ml to about 10 ng/mL following administration.

65. (canceled)

66. A method of treating major depressive disorder (MDD) in a patient in need thereof, the method comprising administering the transmucosal composition of claim 1.

67. The method of claim 66, wherein the administration provides a DMT Tₘₐₓ of about 0.3 h to about 2 h and a DMT Cₘₐₓ of about 0.1 ng/mL to about 10 ng/ml following administration; or
wherein the administration provides a DMT Tₘₐₓ of about 0.3 h to about 2 h and a DMT AUCₗₐₛₜ of about 0.1 ng·h/mL to about 10 ng·h/mL following administration.

68. (canceled)

69. The method of claim 66, wherein the composition is administered buccally; or
wherein the composition is administered once about every about 2 weeks to about 6 months.

70. (canceled)

71. (canceled)

72. The method of claim 66, wherein the administration provides a reduction in the patient's Montgomery-Asberg Depression Rating Scale (MADRS) total score by at least about 20%, at least about 30%, at least about 40%, or at least about 50% compared to prior to the administration.

73. (canceled)

74. (canceled)

75. The method of claim 66, wherein the patient has a MADRS total score of at least about 20 prior to the administration.

76. (canceled)

77. (canceled)

78. (canceled)

79. (canceled)
